(19) United States
(12) Patent Application Publication
(10) Pub. No.: US 2025/0114377 A1
(43) Pub. Date: Apr. 10, 2025

(54) TWO-PRONGED TREATMENT FOR NIEMANN PICK TYPE C DISEASE

(71) Applicant: UNIVERSITY OF NOTRE DAME DU LAC, South Bend, IN (US)

(72) Inventor: Kevin T. VAUGHAN, South Bend, IN (US)

(73) Assignee: UNIVERSITY OF NOTRE DAME DU LAC, South Bend, IN (US)

(21) Appl. No.: 18/865,785

(22) PCT Filed: May 16, 2023
(86) PCT No.: PCT/US2023/022419

§ 371 Date: Nov. 14, 2024

Related U.S. Application Data
(60) Provisional application No. 63/488,105, filed on Mar. 2, 2023.
(60) Provisional application No. 63/364,807, filed on May 17, 2022.

(51) Int. Cl. A61K 31/575; A61K 38/17; A61K 47/10; A61K 47/26; A61K 48/00; A61P 25/28; C12N 15/85

(52) U.S. Cl. CPC A61K 31/575; A61K 38/1709; A61K 47/10; A61K 47/26; A61K 48/005; A61P 25/28; C12N 15/85; C12N 2800/107

(57) ABSTRACT

Niemann-Pick Disease type C (NPC) is a neurovisceral lysosomal lipid storage disorder that has a wide clinical spectrum. The disclosure provides for the use of oxysterols and optionally, a polypeptide that binds to a Stimulator of Interferon Gene (STING) protein, compositions of the same, and methods using the compounds and composition to treat NPC disease.

### RELATED APPLICATIONS

This application claims priority under 35 U.S.C § 119(e) to U.S. Provisional Patent Application No. 63/364,807 filed May 17, 2022, and U.S. Provisional Patent Application No. 63/488,105 filed Mar. 2, 2023, each of which applications are incorporated herein by reference.

### BACKGROUND

Niemann-Pick Disease type C (NPC) is a neurovisceral lysosomal lipid storage disorder that has a wide clinical spectrum. The disease may cause rapid fatality in neonates, or chronic neurodegenerative symptoms in children and adults. It can present hepatosplenomegaly (enlarged liver and spleen) in infants, children, or adults. NPC is characterized by eye movement abnormalities, dysphagia (difficulty in swallowing) and dysarthria (slurred, irregular speech), ataxia (lack of muscle control), and progressive cognitive dysfunction (progressive intellectual decline) leading to dementia. NPC is also associated with impaired intracellular lipid trafficking, including the pathways for cholesterol, leading to lipid accumulation in the liver, spleen, and in the brain.

Based on molecular genetic testing, the cause of NPC has been identified as associated with autosomal recessively inherited loss-of-function mutations in either the NPC1 or NPC2 genes. Niemann-Pick disease type D (NPD), previously and still sometimes used to describe the genetic isolate from Nova Scotia, is now associated with mutations in the NPC1 gene. The two genes regulate cholesterol homeostasis. NPC1 encodes a putative integral membrane protein containing sequence motifs consistent with a role in intracellular transport of cholesterol to post-lysosomal destinations. This protein binds cholesterol at its N-terminal domain and transports cholesterol to late endosomal/lysosomal compartments where they are hydrolyzed and released as free cholesterol. Defects in this gene, which account for 95% of NPC cases, cause over-accumulation of cholesterol and glycosphingolipids in late endosomal/lysosomal compartments. The NPC2 gene encodes a protein containing a lipid recognition domain that binds and transports cholesterol to the NPC1 protein. Mutation in NPC2 accounts for approximately 4% of NPC cases.

The diagnosis of NPC is confirmed by biochemical testing that demonstrates impaired cholesterol esterification and positive filipin staining in cultured fibroblasts. Biochemical testing to detect carrier status is unreliable. Most individuals with NPC have NPC1, caused by mutations in NPC1; fewer than 20 individuals have been diagnosed with NPC2, caused by mutations in NPC2. Molecular genetic testing of NPC1 and NPC2 detects disease-causing mutations in approximately 94% of individuals with NPC.

Treatment options for NPC are limited. Traditional treatment plans use medicines that aim to control or relieve specific symptoms of the disease. Currently, there is no FDA approved medicine that specifically targets NPC. Only one drug, miglustat (N-butyl-deoxynojirimycin), has been approved in Europe and other countries for the treatment of NPC. Therefore, a need still exists for novel and more effective methods of treating NPC. The present disclosure satisfies these needs.

### SUMMARY OF THE INVENTION

Recent work reveals that proteins encoded by genes implicated in Niemann Pick Type C (NPC) disease serve two independent purposes and both of these are impaired in NPC disease. The first function is lysosomal tubulation and cholesterol transport, which we have developed a therapy to correct using oral delivery of the oxysterol 27-hydroxycholesterol. The second is silencing of the pro-apoptotic factor STING through recruitment to lysosomes for degradation. Because the second function is not rescued by oxysterol treatment, we identified minimal sequences in the NPC1 gene capable of rescuing STING silencing. These sequences may be used to generate gene therapy constructs. Combining oxysterol treatment with gene therapy will correct both the defects caused by NPC gene mutations.

Accordingly, the disclosure provides for methods of treating Nieman-Pick Type C (NPC) disease in a subject in need thereof comprising administering to a subject an effective amount of an oxysterol; and a polypeptide that binds to a Stimulator of Interferon Gene (STING) protein; wherein the oxysterol stimulates lysosomal tubulation and the polypeptide sequesters the STING protein to a lysosomal membrane for degradation, thereby treating NPC disease.

In some embodiments, method of treating a subject having Nieman-Pick Type C (NPC) disease comprising administering to the subject an effective amount of a first composition comprising 27-hydroxycholesterol, phosphate buffered saline, polysorbate-80, polyethylene glycol-400, and a penetration enhancer; a second composition comprising a recombinant virus, wherein the recombinant virus comprises a polynucleotide having a nucleotide sequence encoding a polypeptide of Nieman-Pick C1 protein (NPC1) that binds to a Stimulator of Interferon Gene (STING) protein; wherein the first composition is administered orally, and the second composition is administered intravenously, intramuscularly, or subcutaneously, wherein the 27-hydroxycholesterol stimulates lysosomal tubulation and the polypeptide of NPC1 sequesters the STING protein to a lysosomal membrane for degradation, thereby treating the NPC disease.

In other embodiments, a method of treating Nieman-Pick Type C (NPC) disease in a subject comprising administering to the subject an effective amount of 27-hydroxycholesterol and a pharmaceutical acceptable carrier, wherein the 27-hydroxycholesterol stimulates lysosomal tubulation, thereby treating NPC disease, wherein the pharmaceutically acceptable carrier comprises phosphate buffered saline, a penetration enhancer, polysorbate-80, and polyethylene glycol-400.

These and other features and advantages of this invention will be more fully understood from the following detailed description of the invention taken together with the accompanying claims. It is noted that the scope of the claims is defined by the recitations therein and not by the specific discussion of features and advantages set forth in the present description.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1. Identification of STING-binding Domain Using NPC1 Truncation Constructs. Line diagram of NPC1 deletion constructs indicating the regions of the coding sequence (codes on right side of panel) that were deleted by mutagenesis. Δ25_89 indicates deletion of region coding for amino acids 25-89, Δ91_152 indicates deletion of region coding for amino acids 91-152, Δ156_215 indicates deletion of region coding for amino acids 156-215, Δ298_343 indicates deletion of region coding for amino acids 298-343, Δ1171_1261 indicates deletion of region coding for amino acids 1171-1261. ΔChu_2 and ΔChu_3 indicate sequences identified in Chu et al., 2021 containing sequences coding for amino acids 1-24, 612-821, 1267-1278 (ΔChu_2) or 1-24, 1099-1278 (ΔChu_3). In some embodiments, the constructs include eGFP added to the C-terminus of the protein.

FIG. 2. Impact of StARD9 Mutations on Cholesterol Accumulation. Peak filipin fluorescence measurements (scale=1-2500) in cells transfected with Full-length StARD9 (FL), truncated StARD9 expressing amino acids 1-3920 (ΔDouble), or amino acids 1-4483 (ΔStART). All constructs have sequences encoding eGFP fused to the N-terminus. *=p<0.05; **—p<0.005.

FIG. 3. Impact of StARD9 Mutations on Lysosomal Tubulation. The ability of purified LDL to rescue lysosomal tubulation after serum deprivation (LDL) was compared to control media containing serum (cDMEM) or DMEM containing charcoal-stripped serum (SF). Rescue with purified LDL was assessed in cells expressing StARD9 constructs encoding amino acids 1-3920 (ΔDouble+LDL) or amino acids 1-4483 (ΔSTART+LDL). The impact of treatment with cyclodextrin (CD) was assessed in cells expressing StARD9 constructs encoding amino acids 1-3920 (ΔDouble+CD) or amino acids 1-4483 (ΔSTART+CD). StARD9 constructs contained sequences encoding eGFP at the N-terminus. *=p<0.005.

FIG. 4. Rescue of Lysosomal Tubulation with Purified Oxysterols. Measurements of lysosomal tubulation comparing control complete media (Control) to media depleted of LDL (LDL−) and depleted media supplemented with 27-hydroxycholesterol (27-HC), 24-hydroxycholesterol (24-HC) or 25-hydroxycholesterol (25-HC). Rescue with 24-HC (p<0.005) and 25-HC (p<0.0001) was significantly lower that 27-HC.

FIG. 5. Impact of NPC1 and StARD9 Mutations on Rescue of Lysosomal Tubulation by 27-Hydroxycholesterol (27-HC). The ability of 27-HC to rescue lysosomal tubulation after depletion of NPC1 (shRNA-NPC1) or StARD9 (shRNA-STARD9) was compared to cells expressing the NPC1 (I1061T)-eGFP mutant (I1061T) or a eGFP-StARD9 construct expressing amino acids 1-4483 (STARD9-ΔSTART). ***—p<0.005; ns (not significant).

FIG. 6. Summary Cartoon of Sequential Transfer Model. This new model for the function of NPC1 and NPC2 proteins implicates NPC2 in selecting 27-hydroxycholesterol (27-HC) out of the complex mixture of cholesterol and oxysterols in LDL (Step 1) and transferring it to NPC1 (Step 2) for transport across the lysosomal limiting membrane (Step 3). After 27-HC crosses the lysosomal limiting membrane (Step 4), it binds to and stimulates StARD9 (Step 5) which activates lysosomal tubulation.

FIG. 7A-C. Behavioral Analysis of NPC1(−/−) Mice Treated with 27-HC. NPC1(−/−) treated with 0 mg/kg (●) or 10 mg/kg (▴) 27-HC once per week by intraperitoneal injection were tested using (A) Tremor Scores p=0.01), (B) Rotarod Analysis (p=0.0037) or (C) Coathanger Tests (p=0.0229).

FIG. 8. Impact of 27-HC Treatment on NPC1 (−/−) Mice. Immunohistochemical staining of cerebella stained for calbindin after treatment with either Omg/kg or 10 mg·kg 27-HC once per week by intraperitoneal injection. Brown staining indicates Purkinje cell survival in lobes of the cerebellum (I, V, X).

FIG. 9A-C. Impact of 27-HC Treatment on NPC1 (−/−) Mice. Quantification of Purkinje cells preservation at days (A) P42, (B) P63 or (C) P77. Lobes are binned as I-V, VI-VII, VIII, or IX-X. A Student's t-test was calculated on total Purkinje cell counts from each timepoint (p-values).

FIG. 10. Impact of 27-HC Treatment on NPC1 (−/−) Mice. Kaplan-Meier lifespan analysis of NPC1 (−/−) mice treated with either 0 mg/kg (dashed line) or 10 mg/kg (solid line) 27-HC. Extension of lifespan was significant in mice treated with 27-HC (p=0.0214).

FIG. 11. Impact of 27-HC Treatment on NPC1 (I1061T/I1061T) Mice. Kaplan-Meier lifespan analysis of NPC1 (I1061T/I1061T) mice treated with either 0 mg/kg (dashed line) or 5 mg/kg (solid line) 27-HC three times per week by oral gavage. Extension of lifespan was significant in mice treated with 27-HC (p=0.0108).

FIG. 12. Impact of 27-HC Treatment on NPC1 (I1061T/I1061T)/StARD9 (−/−) Mice. Kaplan-Meier lifespan analysis of NPC1 (I1061T/I1061T) mice (solid line) to StARD9(−/−)/NPC1 (I1061T/I1061T) mice (dashed line) each treated with 5 mg/kg 27-HC three times per week by oral gavage. 27-HC treatment was significantly less effective in mice mutant for both NPC1 and StARD9 (p=0.0001), supporting StARD9 as the target for 27-HC.

FIG. 13. Impact of 27-HC Treatment on Lipid Accumulation in Livers of Male and Female Mice. Comparing livers of wild-type vs. NPC1 (I1061T/I1061T) mice, lipid accumulations (black arrows) are apparent in NPC1 mutant mice but not wild-type mice. This is true and both males and females. 27-HC treatment is successful in reducing these lipid accumulations (black arrows) in both males and females, however the dosage needed for this reduction is higher in females (40 mg/kg) than males (10 mg/kg).

### DETAILED DESCRIPTION OF THE INVENTION

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001 or Singleton, et al., Dictionary of Microbiology and Molecular Biology, 2d ed., John Wiley and Sons, New York (1994), and Hale & Markham, The Harper Collins Dictionary of Biology. Harper Perennial, N.Y. (1991). General laboratory techniques (DNA extraction, RNA extraction, cloning, PCR amplification, cell culturing. etc.) are known in the art and described, for example, in Molecular Cloning: A Laboratory Manual, J. Sambrook et al., 4th edition, Cold Spring Harbor Laboratory Press, 2012.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five substituents on the ring.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

This disclosure provides ranges, limits, and deviations to variables such as volume, mass, percentages, ratios, etc. It is understood by an ordinary person skilled in the art that a range, such as “number 1” to “number 2”, implies a continuous range of numbers that includes the whole numbers and fractional numbers. For example, 1 to 10 means 1, 2, 3, 4, 5, . . . 9, 10. It also means 1.0, 1.1, 1.2, 1.3, . . . , 9.8, 9.9, 10.0, and also means 1.01, 1.02, 1.03, and so on. If the variable disclosed is a number less than “number10”, it implies a continuous range that includes whole numbers and fractional numbers less than number10, as discussed above. Similarly, if the variable disclosed is a number greater than “number10”, it implies a continuous range that includes whole numbers and fractional numbers greater than number10. These ranges can be modified by the term “about”, whose meaning has been described above.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein may be suitably practiced in the absence of any element or elements, limitation, or limitations not specifically disclosed herein.

An “effective amount” refers to an amount effective to bring about a recited effect, such as an amount necessary to form products in a reaction mixture. Determination of an effective amount is typically within the capacity of persons skilled in the art, especially in light of the detailed disclosure provided herein. The term “effective amount” is intended to include an amount of a compound or reagent described herein, or an amount of a combination of compounds or reagents described herein, e.g., that is effective to form products in a reaction mixture. Thus, an “effective amount” generally means an amount that provides the desired effect.

A “prophylactically effective amount” of a compound is an amount sufficient to prevent a disease or condition, or one or more symptoms associated with the disease or condition, or prevent its recurrence. A prophylactically effective amount of a compound means an amount of therapeutic agent, alone or in combination with other agents, which provides a prophylactic benefit in the prevention of the disease. The term “prophylactically effective amount” can encompass an amount that improves overall prophylaxis or enhances the prophylactic efficacy of another prophylactic agent.

The terms “genome” and “genomic DNA” refer to the heritable genetic information of a host organism. Said genomic DNA comprises the entire genetic material of a cell or an organism, including the DNA of the bacterial chromosome and plasmids for prokaryotic organisms and includes for eukaryotic organisms the DNA of the nucleus (chromosomal DNA), extrachromosomal DNA, and organellar DNA (e.g., of mitochondria). Preferably, the terms genome or genomic DNA refers to the chromosomal DNA of the nucleus.

The term “promoter” refers to a polynucleotide which directs the transcription of a structural gene to produce mRNA. Typically, a promoter is located in the 5′ region of a gene, proximal to the start codon of a structural gene. If a promoter is an inducible promoter, then the rate of transcription increases in response to an inducing agent. In contrast, the rate of transcription is not regulated by an inducing agent, if the promoter is a constitutive promoter. The term “enhancer” refers to a polynucleotide. An enhancer can increase the efficiency with which a particular gene is transcribed into mRNA irrespective of the distance or orientation of the enhancer relative to the start site of transcription. Usually, an enhancer is located close to a promoter, a 5′-untranslated sequence or in an intron.

“Transgene”, “transgenic” or “recombinant” refers to a polynucleotide manipulated by man or a copy or complement of a polynucleotide manipulated by man. For instance, a transgenic expression cassette comprising a promoter operably linked to a second polynucleotide may include a promoter that is heterologous to the second polynucleotide as the result of manipulation by man (e.g., by methods described in Sambrook et al., Molecular Cloning-A Laboratory Manual, Cold Spring Harbor Laboratory, Cold Spring Harbor, New York, (1989) or Current Protocols in Molecular Biology Volumes 1-3, John Wiley & Sons, Inc. (1994-1998)) of an isolated nucleic acid comprising the expression cassette. In another example, a recombinant expression cassette may comprise polynucleotides combined in such a way that the polynucleotides are extremely unlikely to be found in nature. For instance, restriction sites or plasmid vector sequences manipulated by man may flank or separate the promoter from the second polynucleotide. One of skill will recognize that polynucleotides can be manipulated in many ways and are not limited to the examples above.

In case the term “recombinant” is used to specify an organism or cell, e.g., a microorganism, it is used to express that the organism or cell comprises at least one “transgene”, “transgenic” or “recombinant” polynucleotide, which is usually specified later on.

The terms “orthologues” and “paralogues” encompass evolutionary concepts used to describe the ancestral relationships of genes. Paralogues are genes within the same species that have originated through duplication of an ancestral gene; orthologues are genes from different organisms that have originated through speciation and are also derived from a common ancestral gene.

The terms “operable linkage” or “operably linked” are generally understood as meaning an arrangement in which a genetic control sequence, e.g., a promoter, enhancer or terminator, is capable of exerting its function with regard to a polynucleotide being operably linked to it, for example a polynucleotide encoding a polypeptide. Function, in this context, may mean for example control of the expression, i.e., transcription and/or translation, of the nucleic acid sequence. Control, in this context, encompasses for example initiating, increasing, governing or suppressing the expression, i.e., transcription and, if appropriate, translation. Controlling, in turn, may be, for example, tissue- and/or time-specific. It may also be inducible, for example by certain chemicals, stress, pathogens and the like. Preferably, operable linkage is understood as meaning for example the sequential arrangement of a promoter, of the nucleic acid sequence to be expressed and, if appropriate, further regulatory elements such as, for example, a terminator, in such a way that each of the regulatory elements can fulfill its function when the nucleic acid sequence is expressed. An operably linkage does not necessarily require a direct linkage in the chemical sense. For example, genetic control sequences like enhancer sequences are also capable of exerting their function on the target sequence from positions located at a distance to the polynucleotide, which is operably linked. Preferred arrangements are those in which the nucleic acid sequence to be expressed is positioned after a sequence acting as promoter so that the two sequences are linked covalently to one another. The distance between the promoter and the amino acid sequence encoding polynucleotide in an expression cassette, is preferably less than 200 base pairs, especially preferably less than 100 base pairs, very especially preferably less than 50 base pairs. The skilled worker is familiar with a variety of ways in order to obtain such an expression cassette. However, an expression cassette may also be constructed in such a way that the nucleic acid sequence to be expressed is brought under the control of an endogenous genetic control element, for example an endogenous promoter, for example by means of homologous recombination or else by random insertion. Such constructs are likewise understood as being expression cassettes for the purposes of the invention.

The terms “express,” “expressing,” “expressed” and “expression” refer to expression of a gene product (e.g., a biosynthetic enzyme of a gene of a pathway or reaction defined and described in this application) at a level that the resulting enzyme activity of this protein encoded for or the pathway or reaction that it refers to allows metabolic flux through this pathway or reaction in the organism in which this gene/pathway is expressed in. The expression can be done by genetic alteration of the microorganism that is used as a starting organism. In some embodiments, a microorganism can be genetically altered (e.g., genetically engineered) to express a gene product at an increased level relative to that produced by the starting microorganism or in a comparable microorganism which has not been altered. Genetic alteration includes, but is not limited to, altering or modifying regulatory sequences or sites associated with expression of a particular gene (e.g. by adding strong promoters, inducible promoters or multiple promoters or by removing regulatory sequences such that expression is constitutive), modifying the chromosomal location of a particular gene, altering nucleic acid sequences adjacent to a particular gene such as a ribosome binding site or transcription terminator, increasing the copy number of a particular gene, modifying proteins (e.g., regulatory proteins, suppressors, enhancers, transcriptional activators and the like) involved in transcription of a particular gene and/or translation of a particular gene product, or any other conventional means of deregulating expression of a particular gene using routine in the art (including but not limited to use of antisense nucleic acid molecules, for example, to block expression of repressor proteins).

In some embodiments, a microorganism can be physically or environmentally altered to express a gene product at an increased or lower level relative to level of expression of the gene product unaltered microorganism. For example, a microorganism can be treated with, or cultured in the presence of an agent known, or suspected to increase transcription of a particular gene and/or translation of a particular gene product such that transcription and/or translation are enhanced or increased. Alternatively, a microorganism can be cultured at a temperature selected to increase transcription of a particular gene and/or translation of a particular gene product such that transcription and/or translation are enhanced or increased.

The term “domain” refers to a set of amino acids conserved at specific positions along an alignment of sequences of evolutionarily related proteins. While amino acids at other positions can vary between homologues, amino acids that are highly conserved at specific positions indicate amino acids that are likely essential in the structure, stability, or function of a protein. Identified by their high degree of conservation in aligned sequences of a family of protein homologues, they can be used as identifiers to determine if any polypeptide in question belongs to a previously identified polypeptide family.

The term “motif” or “consensus sequence” or “signature” refers to a short, conserved region in the sequence of evolutionarily related proteins. Motifs are frequently highly conserved parts of domains, but may also include only part of the domain, or be located outside of conserved domain (if all of the amino acids of the motif fall outside of a defined domain).

Specialist databases exist for the identification of domains, for example, SMART (Schultz et al. (1998) Proc. Natl. Acad. Sci. USA 95, 5857-5864; Letunic et al. (2002) Nucleic Acids Res30, 242-244), InterPro (Mulder et al., (2003) Nucl. Acids. Res. 31, 315-318), Prosite (Bucher and Bairoch (1994) (In) ISMB-94; Proceedings 2nd International Conference on Intelligent Systems for Molecular Biology. Altman et al., Eds., pp 53-61, AAAI Press, Menlo Park; Hulo et al., Nucl. Acids. Res. 32:D134-D137, (2004)), or Pfam (Bateman et al., Nucleic Acids Research 30(1): 276-280 (2002); Finn et al., Nucleic Acids Research (2010) Database Issue 38:D21 1-222). A set of tools for in silico analysis of protein sequences is available on the ExPASy proteomics server (Swiss Institute of Bioinformatics (Gasteiger et al., Nucleic Acids Res. 31:3784-3788(2003)). Domains or motifs may also be identified using routine techniques, such as by sequence alignment.

Methods for the alignment of sequences for comparison are well known in the art, such methods include GAP, BESTFIT, BLAST, FASTA and TFASTA. GAP uses the algorithm of Needleman and Wunsch ((1970) J Mol Biol 48: 443-453) to find the global (i.e., spanning the complete sequences) alignment of two sequences that maximizes the number of matches and minimizes the number of gaps. The BLAST algorithm (Altschul et al. (1990) J Mol Biol 215: 403-10) calculates percent sequence identity and performs a statistical analysis of the similarity between the two sequences. The software for performing BLAST analysis is publicly available through the National Centre for Biotechnology Information (NCBI). Homologues may readily be identified using, for example, the ClustalW multiple sequence alignment algorithm (version 1.83), with the default pairwise alignment parameters, and a scoring method in percentage. Global percentages of similarity and identity may also be determined using one of the methods available in the MatGAT software package (Campanella et al., BMC Bioinformatics. 2003 Jul. 10; 4:29. MatGAT: an application that generates similarity/identity matrices using protein or DNA sequences.). Minor manual editing may be performed to optimize alignment between conserved motifs, as would be apparent to a person skilled in the art. Furthermore, instead of using full-length sequences for the identification of homologues, specific domains may also be used. The sequence identity values may be determined over the entire nucleic acid or amino acid sequence or over selected domains or conserved motif(s), using the programs mentioned above using the default parameters. For local alignments, the Smith-Waterman algorithm is particularly useful (Smith T F, Waterman M S (1981) J. Mol. Biol 147(1); 195-7).

Typically, this involves a first BLAST involving BLASTing a query sequence against any sequence database, such as the publicly available NCBI database. BLASTN or TBLASTX (using standard default values) are generally used when starting from a nucleotide sequence, and BLASTP or TBLASTN (using standard default values) when starting from a protein sequence. The BLAST results may optionally be filtered. The full-length sequences of either the filtered results or non-filtered results are then BLASTed back (second BLAST) against sequences from the organism from which the query sequence is derived. The results of the first and second BLASTS are then compared. A paralogue is identified if a high-ranking hit from the first blast is from the same species as from which the query sequence is derived, a BLAST back then ideally results in the query sequence amongst the highest hits; an orthologue is identified if a high-ranking hit in the first BLAST is not from the same species as from which the query sequence is derived, and preferably results upon BLAST back in the query sequence being among the highest hits. High-ranking hits are those having a low E-value. The lower the E-value, the more significant the score (or in other words the lower the chance that the hit was found by chance).

Computation of the E-value is well known in the art. In addition to E-values, comparisons are also scored by percentage identity. Percentage identity refers to the number of identical nucleotides (or amino acids) between the two compared nucleic acid (or polypeptide) sequences over a particular length. In the case of large families, ClustalW may be used, followed by a neighbor joining tree, to help visualize clustering of related genes and to identify orthologues and paralogues.

The term “sequence identity” between two nucleic acid sequences is understood as meaning the percent identity of the nucleic acid sequence over in each case the entire sequence length which is calculated by alignment with the aid of the program algorithm GAP (Wisconsin Package Version 10.0, University of Wisconsin, Genetics Computer Group (GCG), Madison, USA), setting, for example, the following parameters: Gap Weight: 12 Length Weight: 4; Average Match: 2,912 Average Mismatch: −2,003.

The term “sequence identity” between two amino acid sequences is understood as meaning the percent identity of the amino acids sequence over in each case the entire sequence length which is calculated by alignment with the aid of the program algorithm GAP (Wisconsin Package Version 10.0, University of Wisconsin, Genetics Computer Group (GCG), Madison, USA), setting, for example, the following parameters: Gap Weight: 8; Length Weight: 2; Average Match: 2,912; Average Mismatch: −2,003.

The term “hybridization” as defined herein is a process wherein substantially homologous complementary nucleotide sequences anneal to each other. The hybridization process can occur entirely in solution, i.e., both complementary nucleic acids are in solution. The hybridization process can also occur with one of the complementary nucleic acids immobilized to a matrix such as magnetic beads, Sepharose beads or any other resin. The hybridization process can furthermore occur with one of the complementary nucleic acids immobilized to a solid support such as a nitro-cellulose or nylon membrane or immobilized by e.g., photolithography to, for example, a siliceous glass support (the latter known as nucleic acid arrays or microarrays or as nucleic acid chips). In order to allow hybridization to occur, the nucleic acid molecules are generally thermally or chemically denatured to melt a double strand into two single strands and/or to remove hairpins or other secondary structures from single stranded nucleic acids.

The term “stringency” refers to the conditions under which a hybridization takes place. The stringency of hybridization is influenced by conditions such as temperature, salt concentration, ionic strength and hybridization buffer composition. Low stringency conditions are selected to be about 30° C. lower than the thermal melting point (Tₘ) for the specific sequence at a defined ionic strength and pH. Medium stringency conditions are when the temperature is 20° C. below Tₘ, and high stringency conditions are when the temperature is 10° C. below Tₘ. High stringency hybridization conditions are typically used for isolating hybridizing sequences that have high sequence similarity to the target nucleic acid sequence. However, nucleic acids may deviate in sequence and still encode a substantially identical polypeptide, due to the degeneracy of the genetic code. Therefore, medium stringency hybridization conditions may sometimes be needed to identify such nucleic acid molecules.

The Tₘ is the temperature under defined ionic strength and pH, at which 50% of the target sequence hybridizes to a perfectly matched probe. The Tₘ is dependent upon the solution conditions and the base composition and length of the probe. For example, longer sequences hybridize specifically at higher temperatures. The maximum rate of hybridization is obtained from about 16° C. up to 32° C. below Tₘ. The presence of monovalent cations in the hybridization solution reduces the electrostatic repulsion between the two nucleic acid strands thereby promoting hybrid formation; this effect is visible for sodium concentrations of up to 0.4M (for higher concentrations, this effect may be ignored). Formamide reduces the melting temperature of DNA-DNA and DNA-RNA duplexes with 0.6 to 0.7° C. for each percent formamide, and addition of 50% formamide allows hybridization to be performed at 30 to 45° C., though the rate of hybridization will be lowered. Base pair mismatches reduce the hybridization rate and the thermal stability of the duplexes. On average and for large probes, the Tₘ decreases about 1° C. per % base mismatch. The Tₘ may be calculated using the following equations, depending on the types of hybrids:
1) DNA-DNA hybrids (Meinkoth and Wahl, Anal. Biochem., 138: 267-284, 1984):

$${T}_{m} = 81.5 ° C . + 16.6 \times {logio [ {Na}^{+} ]}^{a} + 0.41 \times \% [ G / {C}^{b} ] - 500 \times [ {L}^{c} ] {-}^{1} - 0.61 \times \% formamide$$

2) DNA-RNA or RNA-RNA hybrids:

$${T}_{m} = 79.8 ° C . + 18.5 ( {logio [ {Na}^{+} ]}^{a} ) + 0.58 ( \% G / {C}^{b} ) + 11.8 {( \% G / {C}^{b} )}^{2} - 820 / {L}^{c}$$

3) oligo-DNA or oligo-RNA^{d} hybrids:

$$For < 20 nucleotides : {T}_{m} = 2 ( {l}_{n} )$$

$$For 20 - 35 nucleotides : {T}_{m} = 22 + 1.46 ( {l}_{n} )$$

a: or for other monovalent cation, but only accurate in the 0.01-0.4 M range.
b: only accurate for % GC in the 30% to 75% range.
c: L=length of duplex in base pairs.
d: oligo, oligonucleotide; lₙ, =effective length of primer=2×(no. of G/C)+(no. of A/T).

Non-specific binding may be controlled using any one of a number of known techniques such as, for example, blocking the membrane with protein containing solutions, additions of heterologous RNA, DNA, and SDS to the hybridization buffer, and treatment with RNAse. For non-homologous probes, a series of hybridizations may be performed by varying one of (i) progressively lowering the annealing temperature (for example from 68° C. to 42° C.) or (ii) progressively lowering the formamide concentration (for example from 50% to 0%). The skilled artisan is aware of various parameters which may be altered during hybridization, and which will either maintain or change the stringency conditions.

Besides the hybridization conditions, specificity of hybridization typically also depends on the function of post-hybridization washes. To remove background resulting from non-specific hybridization, samples are washed with dilute salt solutions. Critical factors of such washes include the ionic strength and temperature of the final wash solution: the lower the salt concentration and the higher the wash temperature, the higher the stringency of the wash. Wash conditions are typically performed at or below hybridization stringency. A positive hybridization gives a signal that is at least twice that of the background. Suitable stringent conditions for nucleic acid hybridization assays or gene amplification detection procedures are as set forth above. More or less stringent conditions may also be selected. The skilled artisan is aware of various parameters which may be altered during washing and which will either maintain or change the stringency conditions.

For example, typical high stringency hybridization conditions for DNA hybrids longer than 50 nucleotides encompass hybridization at 65° C. in 1×SSC or at 42° C. in 1×SSC and 50% formamide, followed by washing at 65° C. in 0.3×SSC. Examples of medium stringency hybridization conditions for DNA hybrids longer than 50 nucleotides encompass hybridization at 50° C. in 4×SSC or at 40° C. in 6×SSC and 50% formamide, followed by washing at 50° C. in 2×SSC. The length of the hybrid is the anticipated length for the hybridizing nucleic acid. When nucleic acids of known sequence are hybridized, the hybrid length may be determined by aligning the sequences and identifying the conserved regions described herein. 1×SSC is 0.15M NaCl and 15 mM sodium citrate; the hybridization solution and wash solutions may additionally include 5×Denhardt's reagent, 0.5-1.0% SDS, 100 g/ml denatured, fragmented salmon sperm DNA, 0.5% sodium pyrophosphate.

For the purposes of defining the level of stringency, reference can be made to Sambrook et 30 al. (2001) Molecular Cloning: a laboratory manual, 3^{rd} Edition, Cold Spring Harbor Laboratory Press, CSH, New York or to Current Protocols in Molecular Biology, John Wiley & Sons, N.Y. (1989 and yearly updates).

“Homologues” of a protein encompass peptides, oligopeptides, polypeptides, proteins and enzymes having amino acid substitutions, deletions and/or insertions relative to the unmodified protein in question and having similar biological and functional activity as the unmodified protein from which they are derived.

A “deletion” refers to removal of one or more amino acids from a protein.

An “insertion” refers to one or more amino acid residues being introduced into a predetermined site in a protein. Insertions may comprise N-terminal and/or C-terminal fusions as well as intra-sequence insertions of single or multiple amino acids. Generally, insertions within the amino acid sequence will be smaller than N- or C-terminal fusions, of the order of about 1 to 10 residues. Examples of N- or C-terminal fusion proteins or peptides include the binding domain or activation domain of a transcriptional activator as used in the yeast two-hybrid system, phage coat proteins, (histidine)-6-tag, glutathione S-transferase-tag, protein A, maltose-binding protein, dihydrofolate reductase, Tag^{«}100 epitope, c-myc epitope, FLAG©-epitope, lacZ, CMP (calmodulin-binding peptide), HA epitope, protein C epitope and VSV epitope.

A “substitution” refers to replacement of amino acids of the protein with other amino acids having similar properties (such as similar hydrophobicity, hydrophilicity, antigenicity, propensity to form or break a-helical structures or β-sheet structures). Amino acid substitutions are typically of single residues but may be clustered depending upon functional constraints placed upon the polypeptide and may range from 1 to 10 amino acids; insertions will usually be of the order of about 1 to 10 amino acid residues. The amino acid substitutions are preferably conservative amino acid substitutions. Conservative substitution tables are well known in the art (see for example Creighton (1984) Proteins. W.H. Freeman and Company (Eds).

The term “vector”, preferably, encompasses phage, plasmid, fosmid, viral vectors as well as artificial chromosomes, such as bacterial or yeast artificial chromosomes. Moreover, the term also relates to targeting constructs which allow for random or site-directed integration of the targeting construct into genomic DNA. Such target constructs, preferably, comprise DNA of sufficient length for either homologous or heterologous recombination as described in detail below. The vector encompassing the polynucleotide of the present invention, preferably, further comprises selectable markers for propagation and/or selection in a recombinant microorganism. The vector may be incorporated into a recombinant microorganism by various techniques well known in the art. If introduced into a recombinant microorganism, the vector may reside in the cytoplasm or may be incorporated into the genome. In the latter case, it is to be understood that the vector may further comprise nucleic acid sequences which allow for homologous recombination or heterologous insertion. Vectors can be introduced into prokaryotic or eukaryotic cells via conventional transformation or transfection techniques.

The terms “transformation” and “transfection”, conjugation and transduction, as used in the present context, are intended to comprise a multiplicity of prior-art processes for introducing foreign nucleic acid (for example DNA) into a recombinant microorganism, including calcium phosphate, rubidium chloride or calcium chloride co-precipitation, DEAE-dextran-mediated transfection, lipofection, natural competence, carbon-based clusters, chemically mediated transfer, electroporation or particle bombardment. Methods for many species of microorganisms are readily available in the literature.

### Embodiments of the Invention

Applicant identified a smaller region of NPC1 that can fit in the adenovirus vector and rescue the STING silencing function. Coupling this with 27-hydroxycholesterol (27-HC) treatment rescues both defects in NPC disease. Combining that benefit with a gene therapy for NPC1 constructs encoding the STING silencing domain, this invention has the potential to provide a complete solution to NPC disease. Confidence in the gene therapy side of this approach is boosted by the recent FDA approval of a gene therapy for SMA disease. This one-time treatment delivers the wild-type SMA1 sequence using this same vector system. This is commercially available from Novartis and effective. The sequences in NPC1 disclosed herein are smaller than the SMA sequence in that therapy.

Mutations in the NPC1 gene affect two independent lysosomal functions. The first is the generation of lysosomal tubules as a mechanism to deliver intracellular cholesterol to other membrane-bound compartments. Because this function is impaired in NPC disease, we investigated the protein components necessary for tubule projection. We identified 27-HC as a signaling molecule that stimulates lysosomal tubulation and is not provided properly to lysosomal membrane to elicit tubulation in NPC disease. To correct this defect, we provide pure 27-HC in a cocktail that bypasses endocytosis. This method corrects the lysosomal tubulation defects in cell-based models of NPC disease. We have also tested this therapy in two different transgenic strains of mice with NPC1 mutations. In both cases, therapeutic administration of 27-HC rescues lysosomal tubulation and delays the onset, severity and progression of NPC disease in NPC mutant mice. It also extends the lifespan of mutant mice significantly. Because we know the molecular target for 27-HC (StARD9), we tested 27-HC treatment in mice where we removed the StARD9 gene. The therapy does not provide any benefit to the StARD9 knock-out mice. This confirms the molecular target for 27-HC treatment and is consistent with our proposed model for the defects in NPC disease. Independent of the role for NPC1 in lysosomal tubulation, NPC1 is the “receptor” for the pro-apoptotic protein STING. STING is a signaling molecule that is activated by cell insults, including defective cholesterol transport. Once STING is activated, it triggers programmed cell death pathways. Counteracting STING activation, STING is silenced by binding to lysosomes, which leads to STING degradation and inactivation of this pathway. This silencing is required to prevent STING activation to proceed long-term. We recently discovered that the “receptor” on lysosomes that drives STING silencing is also NPC1. In cells mutant for NPC1, STING silencing is impaired, thereby leading to programmed cell death. As a result, NPC1 mutations cause cell death in two different ways. They cause loss of lysosomal tubulation, and they cause perpetual activation of STING.

Because the regions in the NPC1 protein sequence are limited to subdomains, we have identified the STING-binding sequences. These sequences are designed to rescue STING silencing alone and will be delivered using established gene therapy approaches. Coupling rescue of STING silencing with 27-HC treatment will be used to correct both problems caused by NPC1 mutations and provide significant benefits for NPC disease patients.

Accordingly, the disclosure provides for methods of treating Nieman-Pick Type C (NPC) disease. In some embodiments, a method of treating Nieman-Pick Type C (NPC) disease in a subject in need thereof comprising administering to a subject an effective amount of an oxysterol and a polypeptide that binds to a Stimulator of Interferon Gene (STING) protein, wherein the oxysterol stimulates lysosomal tubulation and the polypeptide sequesters the STING protein to a lysosomal membrane for degradation, thereby treating NPC disease. In other embodiments, the effective amount is a prophylactically effective amount.

Oxysterols are oxidized derivatives of cholesterol. Oxysterols useful for the methods and compositions of the present disclosure may be oxidized derivatives of cholesterol wherein cholesterol is oxidized at any carbon of cholesterol. Oxysterols useful for the methods and compositions of the present disclosure may be substituted with an oxygen-containing group, such as, but not limited to, at least one of a hydroxyl, oxo, alkoxy, epoxy or carboxy group.

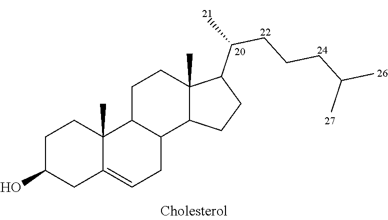 [SMILES: CC(C)CCC[C@@H](C)C1CCC2C3CC=C4C[C@@H](O)CC[C@]4(C)C3CC[C@@]21C]

Oxysterols may be important in many biological processes, including cholesterol homeostasis, atherosclerosis, sphingolipid metabolism, platelet aggregation, apoptosis, and protein prenylation, though their roles are often poorly understood. Oxysterols are lipophilic and cross the blood brain barrier. They are naturally present in small amounts in the brain, and they are known ligands for the Liver X Receptor (LXR) and Sonic Hedgehog (SHH) signaling pathways. Oxysterols may be oxidized at sites on the tetracyclic ring structure or on the C₂₀₋₂₇ aliphatic chain. Specific oxysterols include 3-hydroxy-5-cholesternoic acid, 4-hydroxycholesterol, 7-hydroxycholesterol, 7-hydroperoxycholesterol, 7-ketocholesterol, 24, 25 epoxycholesterol, 20α-hydroxycholesterol, 22(R)-hydroxycholesterol, 22(S)-hydroxycholesterol, 24(R)-hydroxycholesterol, 24(S)-hydroxycholesterol, 25-hydroxycholesterol, and 27-hydroxycholesterol, or a pharmaceutically acceptable salt thereof.

Any oxysterol known in the art may be used in connection with the methods described herein. In certain embodiments, the oxysterol may be selected from the group consisting of 20α-hydroxycholesterol, 22(R)-hydroxycholesterol, 22(S)-hydroxycholesterol; 24(R)-hydroxycholesterol, 24(S)-hydroxycholesterol, 25-hydroxycholesterol, 27-hydroxycholesterol, and a pharmaceutically acceptable salt thereof. In some embodiments, the oxysterol may be selected from the group consisting of 24(R)-hydroxycholesterol, 24(S)-hydroxycholesterol, 25-hydroxycholesterol, 27-hydroxycholesterol, and a pharmaceutically acceptable salt thereof. In some embodiments, the oxysterol is 27-hydroxycholesterol. In some embodiments, the oxysterol is a derivative of an oxysterol described herein. In some embodiments, the oxysterol is a derivative of 27-hydroxycholesterol.

In some embodiments, the oxysterol compound may exist as a stereoisomer wherein asymmetric or chiral centers are present. The stereoisomer is “R” or “S” depending on the configuration of substituents around the chiral carbon atom. The terms “R” and “S” used herein are configurations as defined in IUPAC 1974 Recommendations for Section E, Fundamental Stereochemistry, in Pure Appl. Chem., 45: 13-30 (1976). The disclosure contemplates various stereoisomers and mixtures thereof and these are specifically included within the scope of this invention. Stereoisomers include enantiomers and diastereomers, and mixtures of enantiomers or diastereomers.

Individual stereoisomers of the compounds may be prepared synthetically from commercially available starting materials, which contain asymmetric or chiral centers or by preparation of racemic mixtures followed by methods of resolution well-known to those of ordinary skill in the art. These methods of resolution are exemplified by (1) attachment of a mixture of enantiomers to a chiral auxiliary, separation of the resulting mixture of diastereomers by recrystallization or chromatography, and optional liberation of the optically pure product from the auxiliary as described in Furniss, Hannaford, Smith, and Tatchell, “Vogel's Textbook of Practical Organic Chemistry”, 5th edition (1989), Longman Scientific & Technical, Essex CM20 2JE, England, (2) direct separation of the mixture of optical enantiomers on chiral chromatographic columns, or (3) fractional recrystallization methods.

It should be understood that the oxysterol compound may possess tautomeric forms, as well as geometric isomers, and that these also constitute an aspect of the invention.

The present disclosure also contemplates an isotopically labeled compounds, which is identical to the recited oxysterols, but for the fact that one or more atoms are replaced by an atom having an atomic mass or mass number different from the atomic mass or mass number usually found in nature. Examples of isotopes suitable for inclusion in the compounds of the invention are hydrogen, carbon, nitrogen, oxygen, phosphorus, sulfur, fluorine, and chlorine, such as, but not limited to ²H, ³H, ¹³C, ¹⁴C, ¹⁵N, ¹⁸O, ¹⁷O, ³¹P, ³²P, ³⁵S, ¹⁸F, and ³⁶Cl, respectively.

Substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. The compound may incorporate positron-emitting isotopes for medical imaging and positron-emitting tomography (PET) studies for determining the distribution of receptors. Suitable positron-emitting isotopes that can be incorporated in the disclosed oxysterols are ¹¹C, ¹³N, ¹⁵O, and ¹⁸F.

Isotopically labeled oxysterols can generally be prepared by conventional techniques known to those skilled in the art or by processes analogous to those described in the accompanying Examples using appropriate isotopically labeled reagent in place of non-isotopically-labeled reagent.

In some embodiments, the oxysterol (e.g., 27-hydroxycholesterol) is administered to the subject in a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, or about 50 mg/kg. In some embodiments, the oxysterol (e.g., 27-hydroxycholesterol) is administered to the subject in a dose of about 1 mg/kg to about 20 mg/kg, about 5 mg/kg to about 15 mg/kg, about 8 mg/kg to about 12 mg/kg, or about 10 mg/kg.

In other embodiments, a therapeutically effective amount of an oxysterol disclosed herein may be about 1 mg/kg to about 1000 mg/kg, about 5 mg/kg to about 950 mg/kg, about 10 mg/kg to about 900 mg/kg, about 15 mg/kg to about 850 mg/kg, about 20 mg/kg to about 800 mg/kg, about 25 mg/kg to about 750 mg/kg, about 30 mg/kg to about 700 mg/kg, about 35 mg/kg to about 650 mg/kg, about 40 mg/kg to about 600 mg/kg, about 45 mg/kg to about 550 mg/kg, about 50 mg/kg to about 500 mg/kg, about 55 mg/kg to about 450 mg/kg, about 60 mg/kg to about 400 mg/kg, about 65 mg/kg to about 350 mg/kg, about 70 mg/kg to about 300 mg/kg, about 75 mg/kg to about 250 mg/kg, about 80 mg/kg to about 200 mg/kg, about 85 mg/kg to about 150 mg/kg, about 90 mg/kg to about 100 mg/kg, or a range defined by any two of the foregoing values.

In some embodiments, the polypeptide that binds to a Stimulator of Interferon Gene (STING) protein comprises the NPC1 protein. In some embodiments, the amino acid sequence of NPC1 (e.g., NCBI Reference Sequence No. NP_000262.2) comprises:

(SEQ ID NO: 1)
MTARGLALGLLLLLLCPAQVFSQSCVWYGECGIAYGDKRYNCEYSGPPKPLPKDGYDL
VQELCPGFFFGNVSLCCDVRQLQTLKDNLQLPLQFLSRCPSCFYNLLNLFCELTCSPRQS
QFLNVTATEDYVDPVTNQTKTNVKELQYYVGQSFANAMYNACRDVEAPSSNDKALGL
LCGKDADACNATNWIEYMFNKDNGQAPFTITPVFSDFPVHGMEPMNNATKGCDESVD
EVTAPCSCQDCSIVCGPKPQPPPPPAPWTILGLDAMYVIMWITYMAFLLVFFGAFFAVW
CYRKRYFVSEYTPIDSNIAFSVNASDKGEASCCDPVSAAFEGCLRRLFTRWGSFCVRNP
GCVIFFSLVFITACSSGLVFVRVTTNPVDLWSAPSSQARLEKEYFDQHFGPFFRTEQLIIRA
PLTDKHIYQPYPSGADVPFGPPLDIQILHQVLDLQIAIENITASYDNETVTLQDICLAPLSP
YNTNCTILSVLNYFQNSHSVLDHKKGDDFFVYADYHTHFLYCVRAPASLNDTSLLHDPC
LGTFGGPVFPWLVLGGYDDQNYNNATALVITFPVNNYYNDTEKLQRAQAWEKEFINFV
KNYKNPNLTISFTAERSIEDELNRESDSDVFTVVISYAIMFLYISLALGHMKSCRRLLVDS
KVSLGIAGILIVLSSVACSLGVFSYIGLPLTLIVIEVIPFLVLAVGVDNIFILVQAYQRDERL
QGETLDQQLGRVLGEVAPSMFLSSFSETVAFFLGALSVMPAVHTFSLFAGLAVFIDFLLQ
ITCFVSLLGLDIKRQEKNRLDIFCCVRGAEDGTSVQASESCLFRFFKNSYSPLLLKDWMR
PIVIAIFVGVLSFSIAVLNKVDIGLDQSLSMPDDSYMVDYFKSISQYLHAGPPVYFVLEEG
HDYTSSKGQNMVCGGMGCNNDSLVQQIFNAAQLDNYTRIGFAPSSWIDDYFDWVKPQ
SSCCRVDNITDQFCNASVVDPACVRCRPLTPEGKQRPQGGDFMRFLPMFLSDNPNPKCG
KGGHAAYSSAVNILLGHGTRVGATYFMTYHTVLQTSADFIDALKKARLIASNVTETMGI
NGSAYRVFPYSVFYVFYEQYLTIIDDTIFNLGVSLGAIFLVTMVLLGCELWSAVIMCATIA
MVLVNMFGVMWLWGISLNAVSLVNLVMSCGISVEFCSHITRAFTVSMKGSRVERAEEA
LAHMGSSVFSGITLTKFGGIVVLAFAKSQIFQIFYFRMYLAMVLLGATHGLIFLPVLLSYI
GPSVNKAKSCATEERYKGTERERLLNF.

In some embodiments, the polypeptide comprises NPC1 (NM_000271.5) (cDNA) having a nucleotide sequence: cttcctgaccggcgcgcgcagcctgctgccgcggtcagcgcctgctcctgctcctccgctcctcctgc gcggggtgctgaaacagcccggggaagtagagccgcctccggggagcccaaccagccgaacgccgccggcgtcagcagccttgcgc ggccacagcatgaccgctcgcggcctggcccttggcctcctcctgctgctactgtgtccagcgcaggtgttttcacagtcctgtgtttggtatg gagagtgtggaattgcatatggggacaagaggtaaattgcgaatattctggcccaccaaaaccattgccaaaggatggatatgacttagtgc aggaactctgtccaggattcttctttggcaatgtcagtctctgttgtgatgttcggcagcttcagacactaaaagacaacctgcagctgcctcta cagtttctgtccagatgtccatcctgtttttataacctactgaacctgttttgtgagctgacatgtagccctcgacagagtcagtttttgaatgttac agctactgaagattatgttgatcctgttacaaaccagacgaaaacaaatgtgaaagagttacaatactacgtcggacagagttttgccaatgca atgtacaatgcctgccgggatgtggaggccccctcaagtaatgacaaggccctgggactcctgtgtgggaaggacgctgacgcctgtaat gccaccaactggattgaatacatgttcaataaggacaatggacaggcaccttttaccatcactcctgtgttttcagattttccagtccatgggat ggagcccatgaacaatgccaccaaaggctgtgacgagtctgtggatgaggtcacagcaccatgtagctgccaagactgctctattgtctgtg gccccaagccccagcccccacctcctcctgctccctggacgatccttggcttggacgccatgtatgtcatcatgtggatcacctacatggcgt ttttgcttgtgttttttggagcattttttgcagtgtggtgctacagaaaacggtattttgtctccgagtacactcccatcgatagcaatatagctttttc tgttaatgcaagtgacaaaggagaggcgtcctgctgtgaccctgtcagcgcagcatttgagggctgcttgaggcggctgttcacacgctgg gggtctttctgcgtccgaaaccctggctgtgtcattttcttctcgctggtcttcattactgcgtgttcgtcaggcctggtgtttgtccgggtcacaa ccaatccagttgacctctggtcagcccccagcagccaggctcgcctggaaaaagagtactttgaccagcactttgggcctttettcggacg gagcagctcatcatccgggcccctctcactgacaaacacatttaccagccatacccttcgggagctgatgtaccctttggacctccgcttgac atacagatactgcaccaggttcttgacttacaaatagccatcgaaaacattactgcctcttatgacaatgagactgtgacacttcaagacatctg cttggcccctctttcaccgtataacacgaactgcaccattttgagtgtgttaaattacttccagaacagccattccgtgctggaccacaagaaag gggacgacttctttgtgtatgccgattaccacacgcactttctgtactgcgtacgggctcctgcctctctgaatgatacaagtttgctccatgacc cttgtctgggtacgtttggtggaccagtgttcccgtggcttgtgttgggaggctatgatgatcaaaactacaataacgccactgcccttgtgatt accttccctgtcaataattactataatgatacagagaagctccagagggcccaggcctgggaaaaagagtttattaattttgtgaaaaactaca agaatcccaatctgaccatttccttcactgctgaacgaagtattgaagatgaactaaatcgtgaaagtgacagtgatgtcttcaccgttgtaatta gctatgccatcatgtttctatatatttccctagccttggggcacatgaaaagctgtcgcaggcttctggtggattcgaaggtctcactaggcatc gcgggcatcttgatcgtgctgagctcggtggcttgctccttgggtgtcttcagctacattgggttgcccttgaccctcattgtgattgaagtcatc ccgttcctggtgctggctgttggagtggacaacatcttcattctggtgcaggcctaccagagagatgaacgtcttcaaggggaaaccctgga tcagcagctgggcagggtcctaggagaagtggctcccagtatgttcctgtcatccttttctgagactgtagcatttttcttaggagcattgtccgt gatgccagccgtgcacaccttctctctctttgcgggattggcagtcttcattgactttcttctgcagattacctgtttcgtgagtctcttggggttag acattaaacgtcaagagaaaaatcggctagacatcttttgctgtgtcagaggtgctgaagatggaacaagcgtccaggcctcagagagctgt ttgtttcgcttcttcaaaaactcctattctccacttctgctaaaggactggatgagaccaattgtgatagcaatatttgtgggtgttctgtcattcag catcgcagtcctgaacaaagtagatattggattggatcagtctctttcgatgccagatgactcctacatggtggattatttcaaatccatcagtca gtacctgcatgcgggtccgcctgtgtactttgtcctggaggaagggcacgactacacttcttccaaggggcagaacatggtgtgcggcggc atgggctgcaacaatgattccctggtgcagcagatatttaacgcggcgcagctggacaactatacccgaataggcttcgccccctcgtcctg gatcgacgattatttcgactgggtgaagccacagtcgtcttgctgtcgagtggacaatatcactgaccagttctgcaatgcttcagtggttgac cctgcctgcgttcgctgcaggcctctgactccggaaggcaaacagaggcctcaggggggagacttcatgagattcctgcccatgttcctttc ggataaccctaaccccaagtgtggcaaagggggacatgctgcctatagttctgcagttaacatcctccttggccatggcaccagggtcgga gccacgtacttcatgacctaccacaccgtgctgcagacctctgctgactttattgacgctctgaagaaagcccgacttatagccagtaatgtca ccgaaaccatgggcattaacggcagtgcctaccgagtatttccttacagtgtgttttatgtcttctacgaacagtacctgaccatcattgacgac actatcttcaacctcggtgtgtccctgggcgcgatatttctggtgaccatggtcctcctgggctgtgagctctggtctgcagtcatcatgtgtgc caccatcgccatggtcttggtcaacatgtttggagttatgtggctctggggcatcagtctgaacgctgtatccttggtcaacctggtgatgagct gtggcatctccgtggagttctgcagccacataaccagagcgttcacggtgagcatgaaaggcagccgcgtggagcgcgcggaagaggc acttgcccacatgggcagctccgtgttcagtggaatcacacttacaaaatttggagggattgtggtgttggcttttgccaaatctcaaattttcca gatattctacttcaggatgtatttggccatggtcttactgggagccactcacggattaatatttctccctgtcttactcagttacatagggccatca gtaaataaagccaaaagttgtgccactgaagagcgatacaaaggaacagagcgcgaacggcttctaaatttctagccctctcgcagggcat cctgactgaactgtgtctaagggtcggtcggtttaccactggacgggtgctgcatcggcaaggccaagttgaacaccggatggtgccaacc atcggttgtttggcagcagctttgaacgtagcgcctgtgaactcaggaatgcacagttgacttgggaagcagtattactagatctggaggcaa ccacaggacactaaacttctcccagcctcttcaggaaagaaacctcattctttggcaagcaggaggtgacactagatggctgtgaatgtgatc cgctcactgacactctgtaaaggccaatcaatgcactgtctgtctctccttttaggagtaagccatcccacaagttctataccatatttttagtgac agttgaggttgtagatacactttataacattttatagtttaaagagetttattaatgcaataaattaactttgtacacatttttatataaaaaaacagca agtgatttcagaatgttgtaggcctcattagagcttggtctccaaaaatctgtttgaaaaaagcaacatgttcttcacagtgttcccctagaaagg aagagatttaattgccagttagatgtggcatgaaatgagggacaaagaaagcatctcgtaggtgtgtctactgggttttaacttatttttctttaat aaaatacattgttttcctaagttttggggttaccctatctgctttgagagacaaatacaaaagctaaatggaagaga (SEQ ID NO: 2).

In some embodiments, the STING binding domain comprises a polypeptide comprising amino acids 1-24, and 90-1278 of SEQ ID NO: 1; amino acids 1-90 and 153-1278 of SEQ ID NO: 1; amino acids 1-155 and 216-1278 of SEQ ID NO: 1; amino acids 1-297 and 344-1278 of SEQ ID NO: 1; amino acids 1-1170 and 1262-1278 of SEQ ID NO: 1; amino acids 1-24, 614-821, and 1267-1278 of SEQ ID NO: 1; or amino acids 1-24, and 1099-1278 of SEQ ID NO: 1.

In some embodiments, the STING binding domain consists of a polypeptide comprising amino acids 1-24, and 90-1278 of SEQ ID NO: 1; amino acids 1-90 and 153-1278 of SEQ ID NO: 1; amino acids 1-155 and 216-1278 of SEQ ID NO: 1; amino acids 1-297 and 344-1278 of SEQ ID NO: 1; amino acids 1-1170 and 1262-1278 of SEQ ID NO: 1; amino acids 1-24, 614-821, and 1267-1278 of SEQ ID NO: 1; or amino acids 1-24, and 1099-1278 of SEQ ID NO: 1.

In some embodiments, the STING binding domain consists of a polypeptide consisting of amino acids 1-24, and 90-1278 of SEQ ID NO: 1; amino acids 1-90 and 153-1278 of SEQ ID NO: 1; amino acids 1-155 and 216-1278 of SEQ ID NO: 1; amino acids 1-297 and 344-1278 of SEQ ID NO: 1; amino acids 1-1170 and 1262-1278 of SEQ ID NO: 1; amino acids 1-24, 614-821, and 1267-1278 of SEQ ID NO: 1; or amino acids 1-24, and 1099-1278 of SEQ ID NO: 1.

In some embodiments, the method of treating NPC comprises the use of a recombinant virus comprising a recombinant polynucleotide, wherein the recombinant polynucleotide comprises a nucleotide sequence encoding the polypeptide. In some embodiments, the recombinant virus comprises a lentivirus, an adenovirus, an adeno-associated virus, or a retrovirus. Methods for preparing and using recombinant viruses and recombinant virus vectors are disclosed, for example U.S. Pat. Pub. No. 2022/0001028 to Hatfield et al.; 2022/0125875 to Safieddine et al.; and 2020/0405883 to Bilic et al.

A number of viral and nonviral vectors have been developed for delivery of genetic material in various tissues and organs. In most cases, these vectors are replication incompetent and pose little threat of viral-induced disease. Rather, the viral genome has been partly or fully deleted, expanding the capacity to allow inclusion of therapeutic DNA cargo within the viral capsid. Some vectors include single-stranded DNA, while others include double-stranded DNA. Particularly preferred vectors in the context of the invention are lentiviral vectors, adenovirus vectors, Adeno-associated viruses (AAV) as disclosed in Ahmed et al, JARO 18:649-670 (2017).

The disclosure also provides a vector containing the polynucleotide encoding the STING binding domain polypeptide. In one embodiment, the polynucleotide is operably linked to a promoter. In a further embodiment, the promoter is one of a human cytomegalovirus (CMV) promoter, a CAG promoter, a Rous sarcoma virus (RSV) LTR promoter/enhancer, an SV40 promoter, a EF1-alpha promoter, a CMV immediate/early gene enhancer/CBA promoter, a Nav1.7 promoter, a Nav1.8 promoter, a Nav1.9 promoter, a TRPV1 promoter, a synapsin promoter, a calcium/calmodulin-dependent protein kinase II promoter, a tubulin alpha I promoter, a neuron-specific enolase promoter, a glial fibrillary acidic protein (GFAP) promoter, human NPC1 promoter, and the mouse CaMKII promoter. In some embodiments, the human NPC1 promoter comprises the polynucleotide sequence cgtagaacttaaaacagaccctggtccccacctcggggtttcctcgcgc ccacgggctgcgctgcgcagctgggccagcaggtggccgaggctcccgcactttttttcccgggcctggccgcaccgccaccgcccacc ccgcgctccgccccgccccgccccgccgcactcgcggatcgtcacacggtcaggccaccgcgcgtcaccgccccgggctctgctagaa agtgccggggtccagattcctttcaggtgactgggttgggaggagaagtcgctcacggtaattgtcggagccgaaataaacagagactccg cggccgggcgggaccttacccggaagtcccccagaacacgactacccttcgctgaaacctcgcccgccccctccaacccagagacgaa aagggaaagcatcacggccagaaaccgttggcacaactccacagatactctcccgggccgagccagactccataagtcccgcgcctggc ccccggggattgcaggggctgaggagaagggcaacacggggaccttgaagcggggtcgcggcggcgccccagcccgggccaggga gtcccggcagcggcacctcccagaaagggcggagccgacgacgccttcttccttcctgaccggcgcgcgcagtcctgctgccgcggtca gcgcc (SEQ ID NO: 3). In some embodiments, the mouse CaMKII promoter comprises the polynucleotide sequence acttgtggactaagtttgttcgcatccccttctccaaccccctcagtacatcaccctgggggaacaggg tccacttgctcctgggcccacacagtcctgcagtattgtgtatataaggccagggcaaagaggagcaggttttaaagtgaaaggcaggcag gtgttggggaggcagttaccggggcaacgggaacagggcgtttcggaggtggttgccatggggacctggatgctgacgaaggctcgcg aggctgtgagcagccacagtgccctgctcagaagccccaagctcgtcagtcaagccggttctccgtttgcactcaggagcacgggcaggc gagtggcccctagttctgggggcag (SEQ ID NO: 4).

As used herein, the term “vector” refers to any nucleic acid construct used to transfer a nucleic acid encoding a therapeutic protein into a host cell. In some embodiments, a vector includes a replicon, which functions to replicate the nucleic acid construct. Non-limiting examples of vectors useful for gene therapy include plasmids, phages, cosmids, artificial chromosomes, and viruses, which function as autonomous units of replication in vivo. In some embodiments, a vector is a viral vector for introducing a nucleic acid encoding a therapeutic protein into the host cell.

Many modified eukaryotic viruses useful for gene therapy are known in the art. For example, adeno-associated viruses (AAVs) are particularly well suited for use in human gene delivery because humans are a natural host for the virus, the native viruses are not known to contribute to any diseases, and the viruses illicit a mild immune response.

Specifically, AAVs are small replication-deficient adenovirus-dependent viruses from the Parvoviridae family. They have an icosaedrical capsid of 20-25 nm in diameter and a genome of 4.8 kb flanked by two inverted terminal repeats (ITRs). After uncoating in a host cell, the AAV genome can persist in a stable episome state by forming high molecular weight head-to-tail circular concatamers, or can integrate into the host cell genome. Both scenarios provide long-term and high-level transgene expression. “AAV” or “adeno-associated virus” herein can refer to a virus derived from a naturally occurring “wild-type” AAV genome into which a polynucleotide encoding a therapeutic protein has been inserted, a recombinant virus derived from a recombinant polynucleotide encoding a therapeutic protein packaged into a capsid using capsid proteins encoded by a naturally occurring AAV cap gene, or a recombinant virus derived from a recombinant polynucleotide encoding a therapeutic protein packaged into a capsid using capsid proteins encoded by a non-natural capsid cap gene. Included within the definition of AAV are serotypes AAV type 1 (AAV1), AAV type 2 (AAV2), AAV type 3 (AAV3), AAV type 4 (AAV4), AAV type 5 (AAV5), AAV type 6 (AAV6), AAV type 7 (AAV7), AAV type 8 (AAV8), and AAV type 9 (AAV9) viruses encapsulating a polynucleotide encoding a therapeutic protein and viruses formed by one or more variant AAV capsid proteins encapsulating a polynucleotide encoding a therapeutic protein.

In some embodiments, integration of segment of nucleic acid encoding the STING binding domain protein into in the host genome is possible with the retrovirus, lentivirus, and adeno-associated virus gene transfer methods, often resulting in long term expression of the inserted transgene. Additionally, high transduction efficiencies have been observed in many different cell types and target tissues.

Target population of target cells of a retrovirus can be altered by incorporating foreign envelope proteins. Lentiviral vectors are retroviral vectors that are able to transduce or infect non-dividing cells and typically produce high viral titers. Selection of a retroviral gene transfer system depends on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression, Widely used retroviral vectors include those based upon mouse leukemia virus (MuLV), gibbon ape leukemia virus (GaLV), Simian Immunodeficiency virus (SIV), human immunodeficiency virus (HIV), and combinations thereof (see, e.g., Buchscher, et al. (1992) J. Virol. 66:2731-2739; Johann, et at, (1992) J. Virol. 66:1635-1640; Sommerfelt, et al. (1990) Virol. 176:58-59, Wilson et al. (1989) J. Virol. 63:2374-2378; Miller, et a (1991) J. Virol. 65:2220-2224 (1991) and WO 1994026877 to Wong-Stall et et al.).

As used herein, the term “viral particle” refers to a viral particle encapsulating a polynucleotide encoding a therapeutic protein, which is specific for expression of the therapeutic protein when introduced into a suitable animal host (e.g., a human). Specifically included within the definition of viral particles are recombinant viral particles encapsulating a genome in which a codon-altered polynucleotide, which encodes a therapeutic protein such as a polypeptide comprising a STING binding domain as described herein, has been inserted.

In some embodiments, a method of treating Nieman-Pick Type C (NPC) disease in a subject comprises administering to the subject an effective amount of an oxysterol and a recombinant virus comprising a recombinant polynucleotide, the recombinant polynucleotide comprising a nucleotide sequence encoding a polypeptide that binds to a Stimulator of Interferon Gene (STING) protein, wherein the oxysterol stimulates lysosomal tubulation and the polypeptide sequesters STING protein to a lysosomal membrane for degradation, thereby treating the NPC disease. In some embodiments, for example, the recombinant polynucleotide encodes. In some embodiments, the STING binding domain comprising amino acids 1-24, and 90-1278 of SEQ ID NO: 1; amino acids 1-90 and 153-1278 of SEQ ID NO: 1; amino acids 1-155 and 216-1278 of SEQ ID NO: 1; amino acids 1-297 and 344-1278 of SEQ ID NO: 1; amino acids 1-1170 and 1262-1278 of SEQ ID NO: 1; amino acids 1-24, 614-821, and 1267-1278 of SEQ ID NO: 1; or amino acids 1-24, and 1099-1278 of SEQ ID NO: 1.

In some embodiments, a method of treating Nieman-Pick Type C (NPC) disease in a subject comprises administering to the subject an effective amount of a first composition comprising an oxysterol, a buffering agent, a non-ionic detergent, a polyethylene glycol, and a penetration enhancer; a second composition comprising a recombinant virus, wherein the recombinant virus comprises a recombinant polynucleotide comprising a nucleotide sequence encoding a polypeptide of NPC1 that binds to a Stimulator of Interferon Gene (STING) protein; wherein the first composition is administered orally in an amount of about 8 mg/kg to about 12 mg/kg, and the second composition is administered intravenously, intramuscularly, or subcutaneously; and wherein the oxysterol stimulates lysosomal tubulation and the polypeptide of NPC1 sequesters STING protein to a lysosomal membrane for degradation, thereby treating the NPC disease. In some embodiments, the recombinant virus expresses the polypeptide that binds to a Stimulator of Interferon Gene (STING) protein.

In some embodiments, a method of treating Nieman-Pick Type C (NPC) disease in a subject consists essentially of administering to the subject an effective amount of a first composition consisting essentially of an oxysterol, a buffering agent, a non-ionic detergent, a polyethylene glycol, and a penetration enhancer; a second composition consisting essentially of a recombinant virus, wherein the recombinant virus comprises a recombinant polynucleotide comprising a nucleotide sequence encoding a polypeptide of NPC1 that binds to a Stimulator of Interferon Gene (STING) protein; wherein the first composition is administered orally in an amount of about 8 mg/kg to about 12 mg/kg, and the second composition is administered intravenously, intramuscularly, or subcutaneously; and wherein the oxysterol stimulates lysosomal tubulation and the polypeptide of NPC1 sequesters STING protein to a lysosomal membrane for degradation, thereby treating the NPC disease.

In some embodiments, a method of treating Nieman-Pick Type C (NPC) disease in a subject comprises administering to the subject an effective amount of a first composition comprising an oxysterol, a buffering agent, a non-ionic detergent, a polyethylene glycol, and a penetration enhancer; a second composition comprising a recombinant virus, wherein the recombinant virus comprises a recombinant polynucleotide comprising a nucleotide sequence encoding a polypeptide of NPC1 that binds to a Stimulator of Interferon Gene (STING) protein; wherein the first composition is administered in an amount of about 8 mg/kg to about 12 mg/kg, and the second composition is administered intravenously, intramuscularly, or subcutaneously; and wherein the oxysterol stimulates lysosomal tubulation and the polypeptide of NPC1 sequesters STING protein to a lysosomal membrane for degradation, thereby treating the NPC disease. In some embodiments, the recombinant virus expresses the polypeptide that binds to a Stimulator of Interferon Gene (STING) protein. In some embodiments, the first composition is administered orally, intravenously, intramuscularly, or subcutaneously. In some embodiments, the first composition is administered orally.

In some embodiments, a buffering agent comprises, for example, Tris-Borate-EDTA (TBE) (100 mM Tris-HCl pH 8.0, 90 mM boric acid, and 1 mM EDTA) or TE (10 mM Tris-HCl pH 8.0 and 1 mM EDTA), and phosphate-buffered saline.

In some embodiments, a non-ionic detergent comprises, for example, polyoxyethylene (and related detergents), and glycosidic compounds (e.g., alkyl glycosides). Exemplary alkyl glucosides include octyl β-glucoside, n-dodecyl-β-D-maltoside, beta-decyl-maltoside, and Digitonin. Examples of polyoxyethylene detergents include polysorbates (e.g., polysorbate 20, Polysorbate 40, polysorbate 60, polysorbate 80 (also known as TWEEN-20, TWEEN-40, TWEEN-60, and TWEEN-80, respectively), TRITON-X series (e.g., TRITON X-100), TERGITOL series of detergents (e.g., NP-40), the BRIJ series of detergents (e.g., BRIJ-35, BRIJ-58, BRIJ-L23, BRIJ-L4, BRIJ-010), and PLURONIC F68. Preferably, the non-ionic detergent is a polysorbate, and more preferably, polysorbate 80. Preferably, the non-ionic detergent is present in added to the biological sample to have a final concentration of about 0.1% w/w to about 1% w/w. In certain embodiments, TRITON X-100, polysorbate, or NP-40 are present in a final concentration of about 0.1% w/w to about 1% w/w, or about 0.1% w/w to about 0.5% w/w. In another embodiment, a polysorbate is present in a final concentration of about 1% w/w, about 0.5% w/w, about 0.25% w/w, about 0.15% w/w, or about 0.10% w/w. In one certain embodiment, polysorbate-80 is present in a final concentration of about 0.1% w/w to about 1% w/w, and more preferably, at about 0.1% to about 0.5% w/w.

In some embodiments, the polyethylene glycol (PEG) may have the formula:

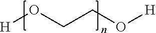

wherein n is 0-10,000. In some embodiments, the PEG is PEG 200 (i.e., n is about 200), PEG 300, PEG 400, PEG 500, PEG 550, PEG 600, PEG 700, PEG 800, PEG 900, PEG 1000, PEG 1450, PEG 3350, PEG 4500, PEG 8000, or combinations thereof.

In some embodiments, a penetration enhancer is present such as an organic solvent that enhances the penetration of an agent in the composition across a biological membrane such as the skin, blood-brain barrier, or a cell plasma membrane as discussed, for example, in Percutaneous Penetration Enhancers, by E. W. Smith and H. I. Maibach, CRC Press, Inc., Boca Raton, Florida, 1995. In certain embodiments, a penetration enhancer includes, but is not limited to, a sulfoxide such as dimethyl sulfoxide (DMSO) or decylmethylsulfoxide, alkanols such as ethanol, propanol, butanol, hexanol, octanol, nonanol, decanol, and 2-butanol, fatty alcohols such as caprylic, decyl, lauryl, myristyl stearyl, and linolenyl alcohol, aliphatic fatty acid esters such as isopropyl n-butyrate, and isopropyl n-hexanoate, alkyl fatty acid esters such as ethyl acetate, methyl acetate, butyl acetate, and methylpropionate, polyols such as glycols, and polyethylene glycol, amides such as dimethylacetamide, dimethylformamide, pyrrolidone and pyrrolidone derivatives, cyclic amides, diethanolamine, and triethanolamine, anionic surfactants such as sodium laurel sulfate and sodium laurate, cationic surfactants such as benzalkonium chloride, cetylpyridinium chloride, and cetyltrimethyl ammonium bromide, non-ionic surfactants such as poloxmer 231, poloxmer 182, BRIJ 30, BRIJ 93, BRIJ 96, TWEEN 20, and TWEEN 80, terpenes such as limonene, carene, pinene, terpinol, carvone, methone, and terpene oxides, alkanones such as n-heptane, n-tetradecane, and organic acids such as salicylic acid, citric acid, and succinic acid.

In some embodiments, the penetration enhancer is DMSO, petrolatum, mineral oil, castor oil, corn oil, glycerol, tocopherol, dimethyl formamide, dihydrolevoglucosenone (i.e., CYRENE), γ-butyrolactone (GBL), N-methyl-2-pyrrolidone (NMP), dimethylacetamide (DMAc), ethanol, or a combination thereof. In some embodiments, the penetration enhancer is DMSO, dimethyl formamide (DMF), dihydrolevoglucosenone (i.e., CYRENE), γ-butyrolactone (GBL), N-methyl-2-pyrrolidone (NMP), dimethylacetamide (DMAc), ethanol, or a combination thereof.

While certain examples described herein employ DMSO as a pharmaceutically acceptable carrier, similar results can be attained using other carriers disclosed herein, for example, a carrier recited in one of the preceding paragraphs.

In some embodiments, the penetration enhancer comprises a polar aprotic solvent. In some embodiments, the polar aprotic solvent as dimethylformamide, dimethylacetamide, N-methyl-2-pyrrolidone, hexamethylphosphoramide (HMPA), or dimethyl sulfoxide (DMSO).

In some embodiments, the oxysterol is orally administered, wherein the oxysterol is admixed with a buffering solution, a penetration enhancer, a polysorbate, and a polyethylene glycol. In some embodiments, the buffering solution comprises phosphate buffered saline, the penetration enhancer is dimethyl sulfoxide, the polysorbate comprises polysorbate-80, and the polyethylene glycol comprises polyethylene glycol-400.

In some embodiments, a composition comprising the oxysterol may comprise, for example, about 1% to about 50% buffering solution, about 1% to about 10% penetration enhancer, about 1% to about 20% polysorbate, and about 1% to about 60% polyethylene glycol. In some embodiments, a composition comprising the oxysterol may comprise, for example, about 10% to about 50% buffering solution, about 1% to about 5% penetration enhancer, about 1% to about 15% polysorbate, and about 10% to about 50% polyethylene glycol. In some embodiments, a composition comprising the oxysterol may comprise, for example, about 45% buffering solution, about 5% penetration enhancer, about 10% polysorbate, and about 40% polyethylene glycol. In some embodiments, a composition comprising the oxysterol may comprise, for example, about 45% buffering solution, about 5% penetration enhancer, about 10% polysorbate, and about 40% polyethylene glycol.

In some embodiments, a composition comprising the oxysterol may comprise, for example, about 45% phosphate buffered saline, about 5% DMSO, about 10% polysorbate-80, and about 40% PEG-400.

In some embodiments, a method of treating Nieman-Pick Type C (NPC) disease in a subject comprises administering to the subject an effective amount of a first composition comprising 27-hydroxycholesterol, phosphate buffered saline, polysorbate-80, polyethylene glycol-400, and penetration enhancer; a second composition comprising a recombinant virus, wherein the recombinant virus comprises a recombinant polynucleotide comprising a nucleotide sequence encoding a polypeptide of NPC1 that binds to a Stimulator of Interferon Gene (STING) protein; wherein the first composition is administered intravenously, intramuscularly, or subcutaneously, in an amount of about 8 mg/kg to about 12 mg/kg, and the second composition is administered intravenously, intramuscularly, or subcutaneously; and wherein the 27-hydroxycholesterol stimulates lysosomal tubulation and the polypeptide of NPC1 sequesters STING protein to a lysosomal membrane for degradation, thereby treating the NPC disease.

In some embodiments, a method of treating Nieman-Pick Type C (NPC) disease in a subject comprises administering to the subject an effective amount of a first composition comprising 27-hydroxycholesterol, phosphate buffered saline, polysorbate-80, polyethylene glycol-400, and a penetration enhancer; a second composition comprising a recombinant virus, wherein the recombinant virus comprises a recombinant polynucleotide comprising a nucleotide sequence encoding a polypeptide of NPC1 that binds to a Stimulator of Interferon Gene (STING) protein; wherein the first composition is administered orally in an amount of about 8 mg/kg to about 12 mg/kg, and the second composition is administered intravenously, intramuscularly, or subcutaneously; and wherein the 27-hydroxycholesterol stimulates lysosomal tubulation and the polypeptide of NPC1 sequesters STING protein to a lysosomal membrane for degradation, thereby treating the NPC disease.

In some embodiments, the oxysterol and the polypeptide, or a first composition comprising the oxysterol and a second composition comprising the polypeptide, are administered sequentially or concurrently. In some embodiments, the polypeptide or the recombinant virus comprising a nucleotide sequence encoding NPC1 or a STING binding domain of NPC1 is administered by injection or intravenous infusion. In some embodiments, oxysterol or a composition comprising the oxysterol is administered orally, via intravenous infusion, or via injection. In some embodiments, oxysterol or a composition comprising the oxysterol is administered orally.

In some embodiments, a method of treating Nieman-Pick Type C (NPC) disease in a subject comprising administering to a subject an effective amount of a composition comprising 27-hydroxycholesterol and a pharmaceutically acceptable carrier, wherein the 27-hydroxycholesterol stimulates lysosomal tubulation, thereby treating the NPC disease.

In some embodiments, a method of treating Nieman-Pick Type C (NPC) disease in a subject comprising administering to the subject an effective amount of 27-hydroxycholesterol and a pharmaceutical acceptable carrier, wherein the 27-hydroxycholesterol stimulates lysosomal tubulation. Optionally, some embodiments may further comprise a polypeptide comprising a STING binding domain of NPC1 as described herein.

In some embodiments, a method of treating Nieman-Pick Type C (NPC) disease in a subject comprising administering to a subject an effective amount of a composition comprising 27-hydroxycholesterol, phosphate buffered saline, a penetration enhancer, polysorbate-80, and polyethylene glycol 400, wherein the 27-hydroxycholesterol stimulates lysosomal tubulation within the target cell, thereby treating the NPC disease. Optionally, some embodiments may further comprise a polypeptide comprising a STING binding domain of NPC1 as described herein.

In some embodiments, a method of treating Nieman-Pick Type C (NPC) disease in a subject comprising administering to a subject an effective amount of a composition comprising 27-hydroxycholesterol, phosphate buffered saline, a penetration enhancer, polysorbate-80, and polyethylene glycol 400, wherein the 27-hydroxycholesterol is present in the composition in an amount of about 5 mg/kg to 20 mg/kg, or optionally about 8 mg/kg to about 12 mg/kg, or optionally, about 10 mg/kg, wherein the 27-hydroxycholesterol stimulates lysosomal tubulation, thereby treating the NPC disease. Optionally, some embodiments may further comprise a polypeptide comprising a STING binding domain of NPC1 as described herein.

In some embodiments, a method of treating Nieman-Pick Type C (NPC) disease in a subject consists essentially of administering to a subject an effective amount of a composition consisting essentially of 27-hydroxycholesterol, phosphate buffered saline, a penetration enhancer, polysorbate-80, and polyethylene glycol 400, wherein the 27-hydroxycholesterol is present in the composition in an amount of about 5 mg/kg to 20 mg/kg, or optionally about 8 mg/kg to about 12 mg/kg, or optionally, about 10 mg/kg, wherein the 27-hydroxycholesterol stimulates lysosomal tubulation, thereby treating the NPC disease. Optionally, some embodiments may further comprise a polypeptide comprising a STING binding domain of NPC1 as described herein.

### Pharmaceutical Formulations

The compounds described herein can be used to prepare therapeutic pharmaceutical compositions, for example, by combining the compounds with a pharmaceutically acceptable diluent, excipient, or carrier. The compounds may be added to a carrier in the form of a salt or solvate. For example, in cases where compounds are sufficiently basic or acidic to form stable nontoxic acid or base salts, administration of the compounds as salts may be appropriate. Examples of pharmaceutically acceptable salts are organic acid addition salts formed with acids that form a physiologically acceptable anion, for example, tosylate, methanesulfonate, acetate, citrate, malonate, tartrate, succinate, benzoate, ascorbate, α-ketoglutarate, andb-glycerophosphate. Suitable inorganic salts may also be formed, including hydrochloride, halide, sulfate, nitrate, bicarbonate, and carbonate salts.

Pharmaceutically acceptable salts may be obtained using standard procedures well known in the art, for example by reacting a sufficiently basic compound such as an amine with a suitable acid to provide a physiologically acceptable ionic compound. Alkali metal (for example, sodium, potassium or lithium) or alkaline earth metal (for example, calcium) salts of carboxylic acids can also be prepared by analogous methods.

The compounds of the formulas described herein can be formulated as pharmaceutical compositions and administered to a mammalian host, such as a human patient, in a variety of forms. The forms can be specifically adapted to a chosen route of enteral administration, e.g., oral administration, sublingual administration, or rectal administration.

The compounds described herein may be systemically administered in combination with a pharmaceutically acceptable vehicle, such as an inert diluent or an assimilable edible carrier. For oral administration, compounds can be enclosed in hard- or soft-shell gelatin capsules, compressed into tablets, or incorporated directly into the food of a patient's diet. Compounds may also be combined with one or more excipients and used in the form of ingestible tablets, buccal tablets, troches, capsules, elixirs, suspensions, syrups, wafers, and the like. Such compositions and preparations typically contain at least 0.1% of active compound. The percentage of the compositions and preparations can vary and may conveniently be from about 0.5% to about 60%, about 1% to about 25%, or about 2% to about 10%, of the weight of a given unit dosage form. The amount of active compound in such therapeutically useful compositions can be such that an effective dosage level can be obtained.

The tablets, troches, pills, capsules, and the like may also contain one or more of the following: binders such as gum tragacanth, acacia, corn starch or gelatin; excipients such as dicalcium phosphate; a disintegrating agent such as corn starch, potato starch, alginic acid and the like; and a lubricant such as magnesium stearate. A sweetening agent such as sucrose, fructose, lactose or aspartame; or a flavoring agent such as peppermint, oil of wintergreen, or cherry flavoring, may be added. When the unit dosage form is a capsule, it may contain, in addition to materials of the above type, a liquid carrier, such as vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propyl parabens as preservatives, a dye and flavoring such as cherry or orange flavor. Any material used in preparing any unit dosage form should be pharmaceutically acceptable and substantially non-toxic in the amounts employed. In addition, the active compound may be incorporated into sustained-release preparations and devices.

Solutions of the active compound or its salts can be prepared in water, optionally mixed with a nontoxic surfactant. Dispersions can be prepared in glycerol, liquid polyethylene glycols, triacetin, or mixtures thereof, or in a pharmaceutically acceptable oil. Under ordinary conditions of storage and use, preparations may contain a preservative to prevent the growth of microorganisms.

Pharmaceutical dosage forms include aqueous solutions, dispersions, or sterile powders comprising the active ingredient, optionally encapsulated in liposomes. The ultimate dosage form should be sterile, fluid and stable under the conditions of manufacture and storage. A liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions, or by the use of surfactants. The prevention of the action of microorganisms can be brought about by various antibacterial and/or antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the compositions can be brought about by agents capable of delaying absorption, for example, aluminum monostearate and/or gelatin.

Various dosage forms can be prepared by incorporating the active compound in the required amount in the appropriate solvent with various other ingredients enumerated above, optionally followed by filter sterilization. Methods of preparation can include vacuum drying and freeze-drying techniques, which yield a powder of the active ingredient plus any additional desired ingredient present in the solution.

Useful solid carriers include finely divided solids such as talc, clay, microcrystalline cellulose, silica, alumina, and the like. Useful liquid carriers include water, dimethyl sulfoxide (DMSO), alcohols, glycols, or water-alcohol/glycol blends, in which a compound can be dissolved or dispersed at effective levels, optionally with the aid of non-toxic surfactants. Adjuvants such as fragrances and additional antimicrobial agents can be added to optimize the properties for a given use. The resultant liquid compositions can be administered orally or sprayed into the mouth using a pump-type or aerosol sprayer. Thickeners such as synthetic polymers, fatty acids, fatty acid salts and esters, fatty alcohols, modified celluloses, or modified mineral materials can also be employed with liquid carriers.

Useful dosages of the compounds described herein can be determined by comparing their in vitro activity, and in vivo activity in animal models. Methods for the extrapolation of effective dosages in mice, and other animals, to humans are known to the art; for example, see U.S. Pat. No. 4,938,949 (Borch et al). The amount of a compound, or an active salt or derivative thereof, required for use in treatment will vary not only with the particular compound or salt selected but also with the route of administration, the nature of the condition being treated, and the age and condition of the patient, and will be ultimately at the discretion of an attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently formulated in unit dosage form; for example, containing 5 to 1000 mg, conveniently 10 to 750 mg, most conveniently, 50 to 500 mg of active ingredient per unit dosage form. In one embodiment, the invention provides a composition comprising a compound of the invention formulated in such a unit dosage form.

The compound can be conveniently administered in a unit dosage form, for example, containing 5 to 1000 mg/m², conveniently 10 to 750 mg/m², most conveniently, 50 to 500 mg/m² of active ingredient per unit dosage form. The desired dose may conveniently be presented in a single dose or as divided doses administered at appropriate intervals, for example, as two, three, four or more sub-doses per day. The sub-dose itself may be further divided, e.g., into several discrete loosely spaced administrations.

The desired dose may conveniently be presented in a single dose or as divided doses administered at appropriate intervals, for example, as two, three, four or more sub-doses per day. The sub-dose itself may be further divided, e.g., into several discrete loosely spaced administrations.

Recombinant viruses or viral vectors may be delivered to a subject in compositions according to any appropriate methods known in the art. The recombinant viruses, preferably suspended in a physiologically compatible carrier (i.e., in a composition), may be administered to a subject, i.e., host animal, such as a human, mouse, rat, cat, dog, sheep, rabbit, horse, cow, goat, pig, guinea pig, hamster, chicken, turkey, or a non-human primate (e.g., Macaque). In some embodiments a host animal does not include a human.

Delivery of the recombinant viruses to a mammalian subject may be by, for example, intramuscular injection or by administration into the bloodstream of the mammalian subject. Administration into the bloodstream may be by injection into a vein, an artery, or any other vascular conduit. In some embodiments, the recombinant viruses are administered into the bloodstream by way of isolated limb perfusion, a technique well known in the surgical arts, the method essentially enabling the artisan to isolate a limb from the systemic circulation prior to administration of the recombinant virus virions. A variant of the isolated limb perfusion technique, described in U.S. Pat. No. 6,177,403, can also be employed by the skilled artisan to administer the virions into the vasculature of an isolated limb to potentially enhance transduction into muscle cells or tissue. Moreover, in certain instances, it may be desirable to deliver the virions to the CNS of a subject. As used herein, “CNS” means all cells and tissue of the brain and spinal cord of a vertebrate. Thus, the term includes, but is not limited to, neuronal cells, glial cells, astrocytes, cerebrospinal fluid (CSF), interstitial spaces, bone, cartilage, and the like. Recombinant AAVs may be delivered directly to the CNS or brain by injection into, e.g., the ventricular region, as well as to the striatum (e.g., the caudate nucleus or putamen of the striatum), spinal cord and neuromuscular junction, or cerebellar lobule, with a needle, catheter or related device, using neurosurgical techniques known in the art, such as by stereotactic injection (see, e.g., Stein et al., J Virol 73:3424-3429, 1999; Davidson et al., PNAS 97:3428-3432, 2000; Davidson et al., Nat. Genet. 3:219-223, 1993; and Alisky and Davidson, Hum. Gene Ther. 11:2315-2329, 2000). In some embodiments, recombinant viruses as described in the disclosure are administered by intravenous injection. In some embodiments, the recombinant viruses are administered by intracerebral injection. In some embodiments, the recombinant viruses are administered by intrathecal injection. In some embodiments, the recombinant viruses are delivered by intracranial injection. In some embodiments, the recombinant viruses are delivered by cisterna magna injection. In some embodiments, the recombinant viruses are delivered by cerebral lateral ventricle injection. In some embodiments, the recombinant viruses are delivered by lumbar puncture injection.

Aspects of the disclosure relate to compositions comprising a recombinant AAV comprising at least one modified genetic regulatory sequence or element. In some embodiments, the composition further comprises a pharmaceutically acceptable carrier.

The compositions of the disclosure may comprise a recombinant virus alone, or in combination with one or more other viruses (e.g., a second recombinant virus encoding having one or more different transgenes). In some embodiments, a composition comprises 1, 2, 3, 4, 5, 6, 7, 8, 9, 10, or more different recombinant viruses each having one or more different transgenes.

Suitable carriers may be readily selected by one of skill in the art in view of the indication for which the recombinant virus is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure.

Optionally, the compositions of the disclosure may contain, in addition to the recombinant viruses and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, parabens, ethyl vanillin, glycerin, phenol, and parachlorophenol. Suitable chemical stabilizers include gelatin and albumin.

Recombinant viruses are administered in sufficient amounts to transfect the cells of a desired tissue and to provide sufficient levels of gene transfer and expression without undue adverse effects. Conventional and pharmaceutically acceptable routes of administration include, but are not limited to, direct delivery to the selected organ (e.g., intraportal delivery to the liver), oral, inhalation (including intranasal and intratracheal delivery), intraocular, intravenous, intramuscular, subcutaneous, intradermal, intratumoral, and other parental routes of administration. Routes of administration may be combined, if desired.

The dose of recombinant viruses virions required to achieve a particular “therapeutic effect,” e.g., the units of dose in genome copies/per kilogram of body weight (GC/kg), will vary based on several factors including, but not limited to: the route of recombinant viruses virion administration, the level of gene or RNA expression required to achieve a therapeutic effect, the specific disease or disorder being treated, and the stability of the gene or RNA product. One of skill in the art can readily determine a recombinant viruses virion dose range to treat a patient having a particular disease or disorder based on the aforementioned factors, as well as other factors that are well known in the art.

An effective amount of recombinant viruses is an amount sufficient to target infect an animal, target a desired tissue. In some embodiments, an effective amount of a recombinant virus is an amount sufficient to produce a stable somatic transgenic animal model. The effective amount will depend primarily on factors such as the species, age, weight, health of the subject, and the tissue to be targeted, and may thus vary among animal and tissue. For example, an effective amount of the recombinant viruses is generally in the range from about 1 ml to about 100 ml of solution containing from about 10⁹ to 10¹⁶ genome copies. In some cases, a dosage between about 10¹¹ to 10¹² recombinant virus genome copies is appropriate. In certain embodiments, 10¹²-10¹³ recombinant virus genome copies are effective to target tissues associated with lysosomal storage diseases, for example brain tissue or CNS tissue. In some cases, stable transgenic animals are produced by multiple doses of recombinant viruses.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1. StARD9 is a Novel Lysosomal Kinesin Required for Membrane Tubulation, Cholesterol Transport, and Purkinje Cell Survival

The following example is described in Sterling et al., J Cell Sci (2023) 136 (5), incorporated herein by reference in its entirety. Late endosomes and lysosomes (LE/Ls) are catabolic organelles in eukaryotes that are responsible for processing materials acquired by endocytosis, autophagy and phagocytosis for utilization and survival. Lysosomal storage diseases (LSDs) are a class of inherited diseases in which one or more components of LE/Ls are. Genetic mutations in catabolic enzyme genes define a majority of LSDs. However, complicating our search for treatments, sometimes the LSD is caused by mutations in genes that do not encode a catabolic enzyme.

Niemann Pick Type C (NPC) disease is a pediatric neurodegenerative disorder caused by the pathological accumulation of cholesterol and other lipids in LE/Ls. Although cholesterol accumulation is apparent in the liver and spleen, the primary problem in NPC disease is the progressive loss of Purkinje cells in the. This loss of Purkinje cells correlates well with the development of ataxia, tremors, slurred speech, loss of eye control and seizures. Although several treatment strategies are under investigation, no treatment for NPC disease has yet received FDA approval.

Two genes (NPC1 and NPC2) are implicated in causing NPC disease. Representing ˜95% of NPC disease cases, NPC1 is a multi-pass transmembrane protein that resides in LE/Ls. It contains an N-terminal cholesterol binding and a transmembrane sterol-sensing domain (SSD). Causing the remaining 5% of cases, NPC2 is a soluble LE/L protein thought to bind.

Although both NPC1 and NPC2 have cholesterol-binding domains, they lack signatures of additional functions. No enzymatic activities have been identified nor indicators of ion binding, nucleotide binding or molecular motor function. The predicted orientation of cholesterol in the binding pockets of NPC1 and NPC2 suggests that the two could exchange cholesterol between each other in a “hand-off” mechanism. Structural studies of NPC1 also suggest that the SSD forms a transmembrane tunnel providing a path for cholesterol across the LE/L limiting membrane. Furthermore, the U18666A drug used to mimic NPC disease blocks this tunnel, providing an explanation for how this drug works and the importance of the SSD. Given the hydrophobic nature of cholesterol, the ultimate destination of this steroid remains unclear once it crosses the LE/L limiting membrane.

Complicating our search for effective therapies, little consensus exists for the specific biological process that leads to lipid accumulation in NPC disease. Although NPC1 and NPC2 both bind cholesterol, the lipid accumulations in NPC disease include cholesterol, oxysterols, sphingolipids, gangliosides, and GPI-anchored proteins. This suggests that some function more global than cholesterol transport alone is impaired. Calcium levels and membrane excitability are also affected. However, neither NPC1 nor NPC2 contain calcium-binding domains. Because mutations in NPC1 fail to concentrate in any one domain, NPC disease has been identified as a protein-folding disease. Thorough analysis of the NPC1 (I1061T) mutation supports this model, implicating loss of protein accumulation as the defect. The impact of NPC1 mutations on the thickness of the glycocalyx in LE/Ls suggests that NPC1 serves a LAMP function as well. Finally, defects in the delivery of cholesterol to the endoplasmic reticulum and plasma membrane suggest a role for NPC proteins in LE/L cholesterol efflux.

In an effort to identify a promising therapy for NPC disease, we sought a global function for NPC1 and NPC2 that could explain the complexities of this disease. We identified LE/L tubulation as a fundamental mechanism of lipid transport that is impaired in NPC disease. We also identified StARD9 as a novel LE/L kinesin responsible for LE/L tubulation. StARD9 colocalizes with NPC1 and induces the same defects in LE/L transport as NPC1 when depleted. Finally, StARD9 KO mice develop the same progressive loss of Purkinje cells and development of neurodegeneration as NPC1 mutant mice. We propose that loss of LE/L tubulation is a significant defect in NPC disease and that NPC1 functions with StARD9 to drive LE/L tubulation.

### NPC Disease and Lysosomal Tubulation:

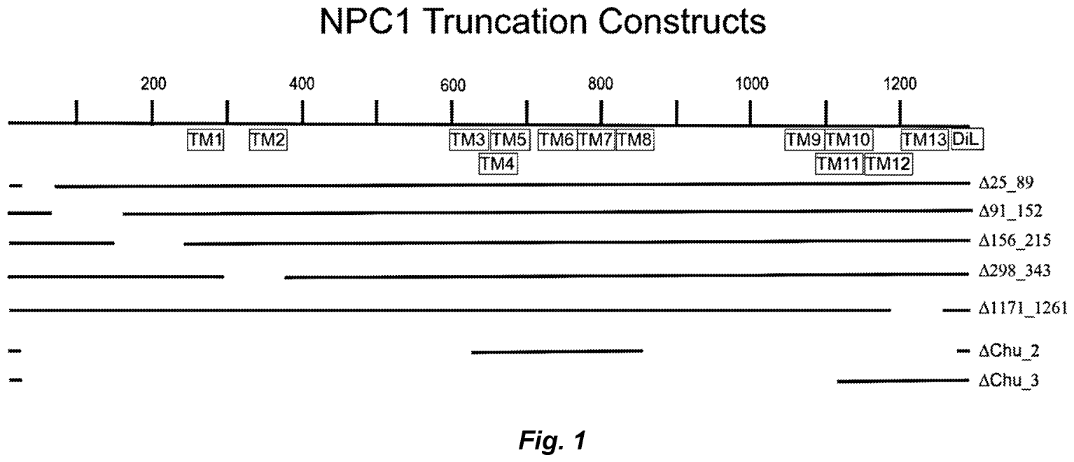

Although it is well-accepted that mutations in NPC1 are responsible for ˜95% of human cases of NPC disease, there is less consensus on the specific cellular activities NPC1 is responsible for. For example, both the N-terminal domain and the sterol-sensing domain of NPC1 are thought to bind cholesterol and mediate the movement of lumenal cholesterol across the limiting membrane of LE/Ls. However, the lipids that over-accumulate in NPC disease are not limited to cholesterol and include oxysterols, sphingolipids, gangliosides and GPI-anchored proteins. Furthermore, the pathological accumulations of cholesterol have been proposed to affect the functional state of LE/Ls directly, including perturbing calcium uptake, autophagy/mitophagy and the thickness of the glycocalyx. However, the specific ways in which NPC1 mutations affect these parameters remain unclear. With this wide range of unknowns in mind, we generated a FP-tagged NPC1 plasmid construct for expression in mammalian cells. As seen previously, the FP-tagged protein encoded by this plasmid accumulated in LE/Ls which clustered near the cell center but also displayed periodic excursions to the cell periphery (See Sterling, FIG. 1A). Also overlapping with previous observations, LE/L membranes incorporating FP-tagged NPC1 projected a population of long, slender, dynamic membrane tubules that emerged from the surface of stationary LE/Ls and extended towards the cell periphery (See Sterling, FIG. 1A and Sup Vid. 1). Tubule projection required microtubules and were inhibited by drugs that interfere with microtubule polymerization and dynamics such as taxol and nocodazole (See Sterling, Sup FIG. 1.1).

To explore the nature of these tubules further, we co-expressed markers of the LE/L membrane or lumenal space with NPC1. NPC1 and LAMP-1 colocalized to the same tubules (See Sterling, FIG. 1A) indicating the presence of a LE/L limiting membrane. NPC1 also colocalized with the soluble LE/L enzyme Cathepsin B (CathB) (See Sterling, FIG. 1A and Sup Vids. 2-4), indicating the presence of a lumenal space within the tubules capable of delivering cholesterol or other lumenal biomolecules. These markers labeled both LE/Ls proper and tubulated projections when expressed alone or in combination (See Sterling, Sup FIG. 1.2). Tubulated LE/L membranes were also detected in fixed cells stained for LE/L proteins (See Sterling, Sup FIG. 1.3). This suggests that the novel structures incorporating NPC1 are hollow, membrane-bounded, tubular membranes.

Because NPC 1 and 2 proteins contain cholesterol binding sites, we imaged NBD-tagged cholesterol with NPC1. This imaging revealed the presence of cholesterol in the tubules, suggesting a potential role for these tubules in cholesterol movement out of LE/Ls proper (See Sterling, Sup FIG. 1.1). Consistent with this prediction, co-expression of markers for NPC1 and the ER suggested contact between LE/L tubules and the branching membranes of the ER (See Sterling, Sup FIG. 1.1). Together these results identify membranous tubule projections as a novel mechanism of cholesterol export from LE/Ls that could be responsible for delivering endocytosed lipids to downstream membranous compartments for utilization.

The most common mutation in human NPC1 is I1061T and is responsible for ˜20% of human NPC disease cases. To compare wild-type and mutant NPC1 in these assays, we expressed a FP-tagged NPC1 (I1061T) construct and performed live-cell imaging. Strikingly, the mutant protein accumulated in LE/L membranes to an extent comparable to wild-type NPC1 (See Sterling, FIG. 1B). However, these membranes failed to project the dynamic membrane tubules observed when using the wild-type construct (See Sterling, Sup Vid. 5). Because the COS-7 cells used for these assays express endogenous, wild-type NPC1, we performed shRNA/rescue to deplete the native NPC1 protein and allow our focus on the FP-tagged proteins (See Sterling, FIG. 1B). ShRNA/rescue with wild-type NPC1 mimicked the previous experiments and revealed projection of dynamic LE/L tubules (See Sterling, FIG. 1B). In contrast, shRNA/rescue with NPC1 (I1061T) produced large spherical LE/Ls of a diameter that suggested engorged lumens (See Sterling, FIG. 1B). These mutant LE/Ls were less motile and failed to project any LE/L tubules. This suggests that LE/Ls incorporating mutant NPC1 are defective in tubule projection, potentially impairing export of endocytosed cholesterol and other biomolecules from LE/Ls to other compartments.

Considering the possible destinations for LE/L cholesterol, we used the ER/nuclear localization of Steroid Response Element Binding Protein (SREBP) to assess LE/L-to-ER transport. Previous work by others reveals that SREBP is a cholesterol sensor that remains a transmembrane protein in the ER membrane if LDL-derived cholesterol is transported successfully from LE/Ls to the ER. However, under conditions that block LE/L-to-ER transport of cholesterol, SREBP undergoes proteolytic cleavage and nuclear import to drive transcription of genes needed for de novo cholesterol biosynthesis. Therefore, the cellular location of SREBP can be used as an indicator of LE/L-to-ER cholesterol transport. SREBP imaging in cells expressing wild-type NPC1 (i.e., COS-7 cells) reveals the typical reticular ER localization pattern with some ER accumulation around the nuclear envelope (See Sterling, FIG. 1C). This indicates retention of SREBP in the ER and successful LE/L-to-ER transport of cholesterol. In contrast, cells homozygous for NPC1 (I1061T) display a strikingly different pattern. The reticular ER localization for SREBP was lost, and SREBP accumulated extensively in the nucleus (See Sterling, FIG. 1C). This indicates a lack of LE/L-to-ER transport, potentially because of a loss of LE/L tubulation.

Overall, these experiments suggest that NPC1 is a component of LE/L tubulation and that this activity is impaired by mutations in NPC1. Furthermore, the loss of LE/L tubulation reduces transport of LDL-derived cholesterol from LE/Ls to the ER, potentially inducing the pathological accumulation of cholesterol and other lipids in LE/Ls that defines NPC disease.

### Identification of StARD9 as a LE/L Kinesin:

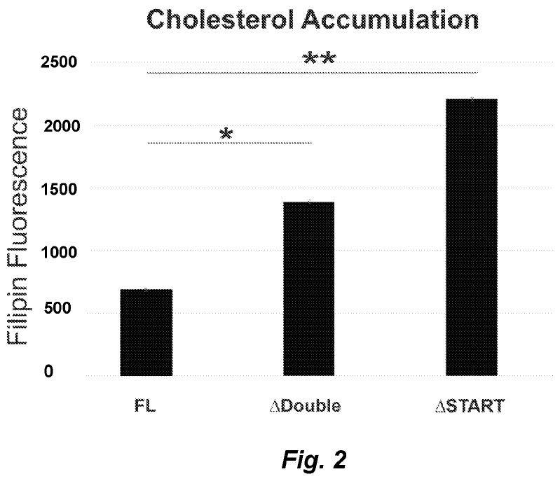

LE/L tubulation is recognized in many cell types, including macrophages where tubulation contributes to formation of phagocytic compartments and elimination of pathogens. Several molecules are implicated in tubule formation. However, current models for tubule formation do not fully-explain the behaviors we observed for NPC1-containing membranes, including vectorial, microtubule-dependent tubule projection and rapid redirection along linear elements. To identify candidates for the dynamic tubule projections we observed, we generated a stable cell line (COS-7) expressing FP-tagged wild-type NPC1. Intact (i.e., non-extracted) NPC1-containing membranes were purified by membrane flotation and immunoprecipitation (See Sterling, Sup FIG. 2A) to complete a proteomic survey of the cytoplasmic face of these LE/Ls. Tryptic fragment data was used to identify candidates, focusing on proteins that shared some type of microtubule binding, some connection to cholesterol and some indication of LE/L localization.

A number of microtubule motor proteins, including cytoplasmic dynein, were identified among the large list of candidates. Interestingly, one candidate emerged in the list that met each of our criteria. StARD9 was identified by 15 tryptic peptides spread across the predicted 4700 amino acid sequence suggested by in silico analysis (See Sterling, FIG. 2 and Sup FIG. 2B). Among the interesting features is an N-terminal kinesin domain (KIF16) and a C-terminal cholesterol-binding domain (StARD) that defines the StART family (See Sterling, FIG. 2A). Although partial sequences for the N-terminus or C-terminus for StARD9 have been cloned independently, full-length cDNA cloning efforts have lagged for StARD9, resulting in some confusion in the literature about the identity and cell biology of this protein. Furthermore, a previous StARD9 study reports an important role for StARD9 in mitotic spindle formation, which appears different from a role in LE/L function.

To resolve some of these important issues, we undertook the complete cloning of StARD9 from human embryonic mRNA. Sequences encoding the C-terminal ⅓ of StARD9 were obtained from existing EST projects, whereas sequences encoding the N-terminal and middle portions of the sequence were cloned by RT-PCR. Using sequence overlap, we assembled a complete sequence encoding the ˜14 kb cDNA (See Sterling, FIG. 2A/B) producing a full-length StARD9 construct that can be used for subsequent analysis.

Our StARD9 sequence encodes each of the 15 tryptic peptides we identified originally and the signature motor domain sequence of KIF16B (See Sterling, Sup FIG. 2C) used previously to define the KIF family. A 60 bp deletion in our sequence near the N-terminus differs from the in silico predictions in Genbank (See Sterling, FIG. 2B). We also identified a conserved DiLeucine (DiL) signal near the C-terminus that is shared with NPC1 and other LAMPs as a LE/L targeting signal (See Sterling, FIG. 2B/C). Together, these data confirm the N-terminal kinesin domain and C-terminal StART domain but also identify a C-terminal LE/L targeting signal in StARD9. This suggests that StARD9 is a previously unknown LE/L kinesin in addition to potential roles in mitotic spindle formation.

Using our constructs for full-length StARD9, we generated new FP-tagged version for expression in mammalian cells for the first time. These expression experiments revealed prominent accumulation of StARD9 in membranes that are also positive for Lysotracker and display typical bidirectional saltatory motion in live-cell imaging (See Sterling, FIG. 2D and Sup Vids. 6-8). This suggests that StARD9 is a LE/L microtubule motor protein that mirrors NPC1 localization during interphase. To assess where StARD9 accumulates during mitosis, we compared constructs encoding the motor domain only to full-length StARD9 (See Sterling, Sup FIG. 2E). The motor-domain construct labeled the mitotic spindle prominently). In contrast, full-length StARD9 labeled membranes that contained LAMP-1 during early and late stages of mitosis.

To explore the potential mechanisms of LE/L accumulation, we performed proteomic MS/MS analysis of latex bead-loaded phagosomes subjected to different extractions. Latex bead-loaded phagosomes provide a biochemically-pure population of NPC1 and LAMP-1-containing membranes with significant advantages over other preparations (Blocker et al. J. Cell Biol. 137, 113-129 (1997)). These membranes were subjected to incubation with: 1) PBS, 2) 1M NaCl, 3) 200 mM Na-Carbonate (pH 11) or 4) 1% Tx-100/1% SDS followed by MRM analysis of tryptic fragments of LAMP-1, NPC1 and StARD9. MRM analysis was used as a detection method because of its significant quantitative and dynamic range advantages over western blot analysis (Li et al., Anal. Chem. 84, 6116-6121 (2012)). Similar to LAMP1 and NPC1, StARD9 was retained in membranes incubated with: 1) PBS, 2) 1M NaCl and 3) 200 mM Na-Carbonate but not in membranes extracted with Tx-100/SDS (See Sterling, FIG. 2E and Table 1). StARD9 detection levels after Tx-100/SDS extraction were more than 10,000-fold lower than the other three conditions, comparable to the results for NPC1 and LAMP1. This suggests a robust mechanism of membrane association for StARD9, potentially important for LE/L tubulation.

Proteins synthesized in the secretory pathway often display N-linked glycosylation, so another indication of membrane association is sensitivity to PNGase F digestion. Using cerebellum tissue as a protein source, we compared control and PNGase-digested samples by western blot analysis. NPC1 displayed a reduction in band complexity and an increase in gel mobility following PNGaseF treatment, indicating N-linked glycosylation for normal samples (See Sterling, FIG. 2F). Although significantly larger than NPC1, StARD9 also displayed a reduction in band complexity and increased gel mobility following PNGaseF treatment (See Sterling, FIG. 2F). Together, these findings suggest that StARD9 undergoes N-linked glycosylation similar to NPC1 and other proteins with membrane association.

### Requirement for StARD9 in LE/L Membrane Mobility:

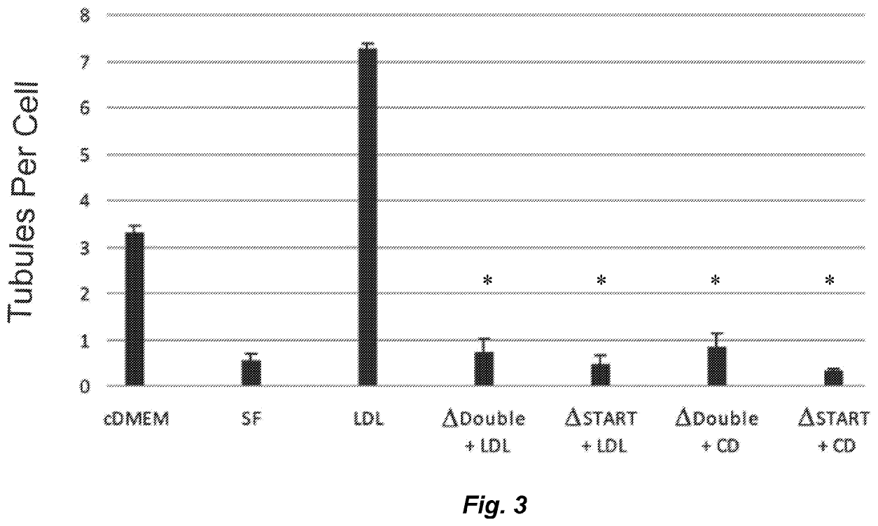

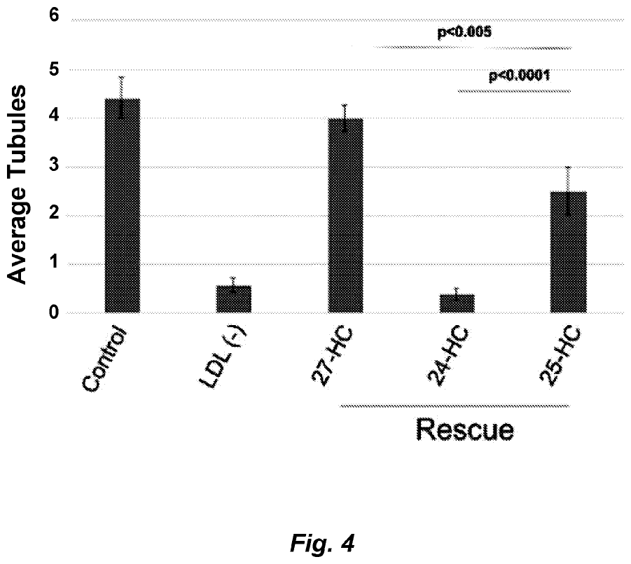

Proposing that StARD9 is a novel LE/L kinesin responsible for LE/L tubulation, we considered several predictions for StARD9 activity. First, if StARD9 and NPC1 participate in the same tubulation process, they should reside in the same membranes. Taking advantage of our cloning of full-length StARD9, we co-expressed StARD9 and NPC1 for live-cell imaging. Consistent with our model, StARD9 and NPC1 accumulated in the same LE/L membranes and incorporated into the same LE/L tubules that projected from the LE/L surface (See Sterling, FIG. 3A-C and Sup Vids. 9-11). To test the requirement for StARD9 in tubulation, we used shRNA to deplete endogenous StARD9 (See Sterling, Sup FIG. 3) and assessed LE/L tubulation of NPC1-containing membranes. StARD9 depletion reduced LE/L tubulation significantly (See Sterling, FIG. 3D-F), suggesting an important role for StARD9 in the projection of membrane tubules. However, when we assessed the overall motility of LE/Ls, we also observed a general impairment of LE/L motility as indicated by kymograph analysis and quantitative analysis of individual membrane motility (See Sterling, FIG. 4A/B). Depletion of StARD9 reduced the number of membrane excursions towards the cell periphery (per 100 secs.), the percent of membranes than underwent excursions (per 100 secs.), and the run-length of excursions using either lysotracker or endocytosed fluorescent dextran as the membrane marker.

Kymograph analysis suggests the immobilization of most LE/L membranes after StARD9 depletion (See Sterling, FIG. 4A/B). To assess relevance for cholesterol transport, we depleted COS-7 cells of StARD9 by shRNA and then measured the impact on cholesterol accumulation in LE/Ls (See Sterling, FIG. 4C). Similar to NPC1 mutations, StARD9 depletion induced a significant increase in peak cholesterol accumulation as indicated by filipin-labeling (See Sterling, FIG. 4C). Finally, depletion of StARD9 by shRNA also drove an accumulation of SREBP in the nucleus, indicating a loss of LE/L-to-ER cholesterol transport (See Sterling, FIG. 1C). This suggests that StARD9 plays a significant role in LE/L behavior overall, including motility, tubulation and cholesterol trafficking.

Using the degree of perinuclear clustering as an indicator of excursions of LE/Ls to the cell periphery, we tested the requirement for StARD9 activity. Cells expressing the non-targeting (N.T.) shRNA displayed ˜65% perinuclear accumulation of lysosomes (See Sterling, FIG. 4D-F), whereas perinuclear accumulation of lysosomes increased to 77% in cells depleted of StARD9 (See Sterling, FIG. 4D-F). This suggests a requirement for StARD9 in the periodic excursions of LE/Ls to the cell periphery. Rescue of StARD9 shRNA depletion with a full-length shRNA-resistant wild-type StARD9 construct shifted the perinuclear accumulation back to ˜66% (See Sterling, FIG. 4D-F), reflecting a return to excursions into the cell periphery. We generated a P-loop mutation (Silvanovich et al., Mol. Biol. Cell 14, 1355-1365 (2003)) in StARD9 that impaired microtubule binding (See Sterling, FIGS. 4D-F and Sup FIG. 4) to assess a requirement for microtubule-dependent motility by StARD9. Rescue of StARD9 shRNA depletion with a full-length shRNA-resistant P-loop mutant construct revealed substantial perinuclear accumulation (See Sterling, FIGS. 4D-F and Sup Vid. 12) comparable to StARD9 depletion alone, suggesting a loss of excursions into the periphery. Together these results suggest that StARD9 is required for motility from the perinuclear region to the cell periphery in addition to a role in LE/L tubulation.

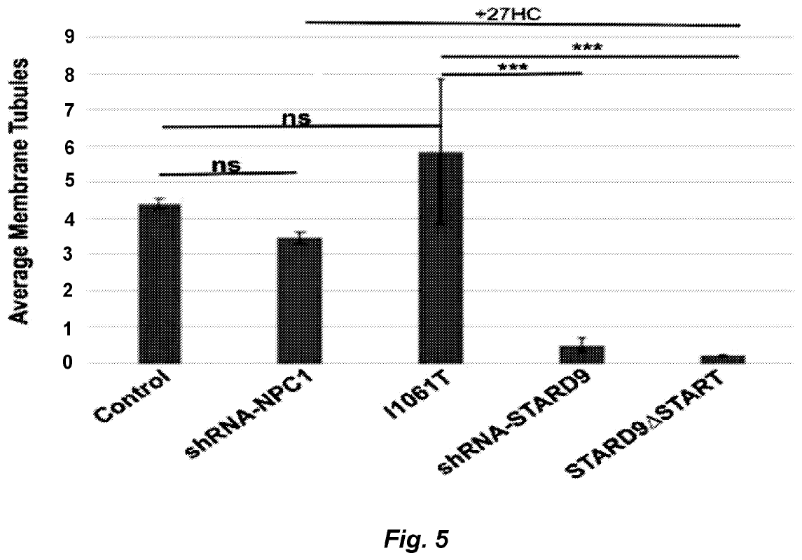

Because we observed a loss of LE/L tubulation in cells expressing mutant NPC1, we investigated how NPC1 mutations might affect StARD9 functionality. As an initial indicator of changes in LE/L proteomics, we generated stable cell lines expressing FP-tagged NPC1 (I1061T) and isolated LE/L membranes as described above for wild-type NPC1. Proteomic mapping studies of these membranes failed to identify StARD9, suggesting some difference in membrane protein composition (data not shown). Because wild-type StARD9 colocalized extensively with wild-type NPC1 (See Sterling, FIG. 3), we repeated the same analysis for the NPC1 (I1061T) mutant construct (See Sterling, FIGS. 5A-C). Interestingly, we observed a significantly-lower level of colocalization from ˜95.% (wild-type NPC1) to ˜65% (NPC1 (I1061T) (See Sterling, FIGS. 5A-C). This suggests that mutations in NPC1 potentially affect the generation of LE/Ls with both NPC1 and StARD9, thereby impairing the ability to project membrane tubules and mediate bidirectional motility.

### CRISPR/CAS9 Removal of StARD9 Induces NPC Disease:

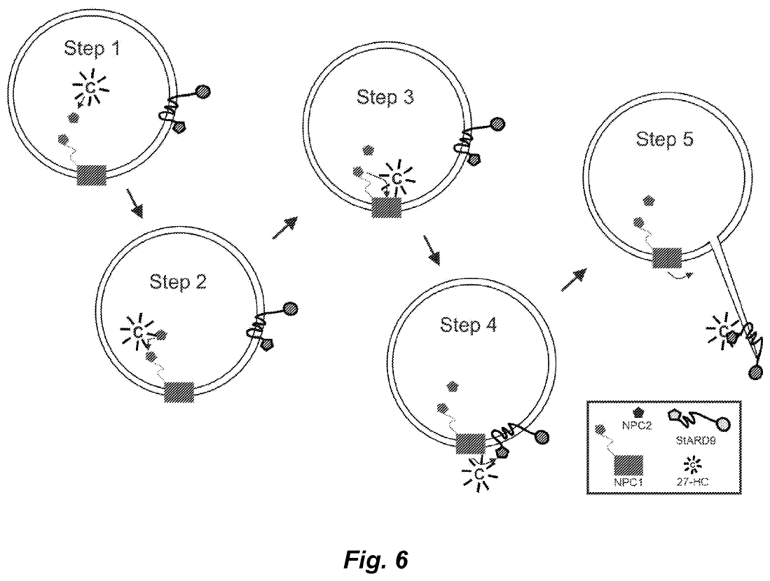

A signature feature of NPC disease is the progressive loss of Purkinje cells in the cerebellum that correlates with the onset and severity of neurodegeneration symptoms. To test if StARD9 is required for the same Purkinje cell survival as NPC genes, we generated StARD9 knock-out mouse lines using CRISPR/CAS-9 excision in 1-cell mouse embryos. 10 mutant mouse lines were produced from 50 embryos (See Sterling, Sup FIG. 6), indicating a significantly better success rate than previous methods of producing knock-out mice. Some of the new mouse lines shared the same sequences surrounding the guide RNA targets, rendering the tracking of specific lineages by genotyping impossible. As a result, we expanded lines with unique genotyping sequences for further analysis (See Sterling, Sup FIG. 6).

StARD9 KO mice develop comparable neurodegeneration phenotypes to NPC mutant mice, with loss of Purkinje cells (See Sterling, FIGS. 6A-E) and the development of tremors, ataxia, loss of grip strength and abnormal walking gait (See Sterling, Table 2). The onset of symptoms was closer to NPC1(I1061T) mutant mice than NPC1 (−/−) mice. However, the pattern of phenotype presentation and the progression towards mortality was analogous.

Consistent with our understanding of NPC disease, StARD9 KO mice displayed a progressive loss of Purkinje cells (See Sterling, FIGS. 6A-E). Purkinje cell loss initiated in lobes 1-2 of the cerebellum but then advanced towards lobe 9 in a progressive pattern over time. Mimicking NPC disease, this correlated with the development of more severe symptoms of neurodegeneration (See Sterling, Table 2). Within individual lobes of the cerebellum, the loss of Purkinje cells appeared somewhat stochastic, with greater loss along the lateral edges than the apical tips (See Sterling, FIGS. 6D/E). This pattern progressed over time towards the more distal lobes. Overall, loss of StARD9 mimics the neurodegenerative features of NPC disease. This suggests that StARD9 is intimately involved in whatever process NPC genes are responsible for.

Mutations in NPC1 have been implicated in causing ˜95% of human Niemann Pick Type C disease cases. However, the mechanisms by which NPC1 mutations impair function remain controversial. A number of consequences of NPC mutations have been reported, including changes in LE/L calcium content, ineffective cholesterol export to the plasma membrane, and a reduction in the thickness of the glycocalyx. Comparing membranes incorporating wild-type vs. I1061T mutant NPC1, we propose that a significant consequence of NPC1 mutations is loss of lysosomal tubulation. Motility differences in membranes containing NPC1 have been reported by others. However, Zhang and coworkers attribute these changes to excessive cholesterol and Ko and coworkers implicate overall motility of LE/Ls proper. An important advance in this study is the potential impact of NPC1 mutations on the projection of lysosomal tubules from the surface of LE/Ls. Because it appears that LE/L tubulation provides a vectorial and deliberate mechanism of intracellular cholesterol delivery, loss of tubulation could explain both the pathological accumulation of cholesterol in NPC disease LE/Ls and the apparent inability to delivery cholesterol to the ER for proper sensing. It could also explain the aberrant mTORC1 signaling detected in NPC disease. Loss of LE/L tubulation could also provide the basis for pathological accumulation of the many lipids beyond cholesterol (oxysterols, sphingolipids, gangliosides and GPI-anchored proteins) that typify NPC disease.

Neither NPC1 nor NPC2 contain the features one might expect for LE/L tubulation. A search for additional components of this pathway identified StARD9 as a previously unknown LE/L kinesin. StARD9 contains a DiL signal, a signature feature of LAMPs and shared with NPC1. Expression of full-length StARD9 reveals accumulation in LE/Ls. Furthermore, shRNA-based depletion of StARD9 reduced LE/L tubulation to an extent comparable to NPC1 mutations. Interestingly, analysis of StARD9 in the Human Protein Atlas project reveals prominent expression in Purkinje cells that suggests lysosomal accumulation. Given the extensive work on other motor proteins implicated in LE/L function, it is surprising that StARD9 was not identified as a candidate previously. Human StARD9 is most similar to members of the KIF16 family (mouse nomenclature). However, the identities of members of the KIF16 protein family were suggested before cloning of these genes was complete.

A comparison of the motor domain of StARD9 to these proteins reveals 93.7% conservation with KIF16B but 63.3% conservation with KIF16A (See Sterling, Sup FIG. 2C). This suggests that StARD9 corresponds to KIF16B rather than KIF16A. Interestingly, previous northern blot analysis suggests that KIF16B labels a very large mRNA, comparable to the StARD9 mRNA (˜-16 kb). In contrast, KIF16A labels a ˜5.2 kb mRNA suggesting a more traditionally sized kinesin (See Sterling, Sup FIG. 2D). Furthermore, the StARD9 gene in humans is located on chromosome 15q15.2 encoding a 4700 A.A. protein with 36 exons. This locus is syntenic to KIF16B mouse chromosome 2.2EF which encodes a 4585 A.A. protein containing 39 exons (See Sterling, Sup FIG. 2D). In contrast, the human KIF16A gene is on chromosome 20p12.1 and encodes a 1317 A.A. protein with 29 exons. This is syntenic to mouse KIF16A which is on chromosome 2.2G1 and encodes a 1312 A.A. protein with 29 exons (See Sterling, Sup FIG. 2D). These and other aspects of StARD9 database annotations could have contributed to the absence of StARD9 in proteomic and other screens of LE/L motor proteins.

Unlike other members of the kinesin family, StARD9 displays a robust association with LE/L membranes. Like LAMPs, StARD9 contains a conserved DiL signal near the C-terminus (See Sterling, FIG. 2B/C). This consensus element is thought to drive retrieval of LAMPs from the plasma membrane for delivery to LE/Ls. Differential extraction experiments reveal that StARD9 can be removed from membranes by detergent, but not by high salt or alkaline pH. This suggests that StARD9 is not a peripheral membrane protein. Finally, western blot analysis of StARD9 after PNGaseF digestion suggests that StARD9 is subject to N-linked glycosylation similar to NPC1. This feature suggests synthesis in the secretory pathway and exposure to the lumenal side of membranes. With a kinesin domain at the N-terminus that is presumably on the cytoplasmic side of LE/L membranes and N-linked glycosylation elsewhere in the molecule, StARD9 mimics bona fide membrane proteins. However, a complete topology map of StARD9 will be required to establish this feature.

Previous work suggests that StARD9 plays a role in spindle pole assembly during mitosis. Although this appears quite different from a role in LE/L motility, our studies do not eliminate the possible role in mitotic spindle function. Perhaps the post-mitotic nature of neurons emphasizes the LE/L function for StARD9 over mitotic functions.

We propose that LE/L tubulation could provide a mechanism to deliver LE/L-associated cholesterol to downstream membranes such as the ER and mitochondria. Tubulated membranes are already described for ER-Golgi transport, TGN membrane transport and endocytic membranes. A feature of tubulated membranes is the combination of lipid bilayer and lumenal space which can contact and exchange with downstream membranes. The lipids known to be over-accumulated in NPC disease include cholesterol, oxysterols, sphingolipids, gangliosides and GPI-anchored proteins. LE/L tubules have the potential to drive the efflux of all these lipids from LE/Ls. Furthermore, the loss of tubulation caused by NPC mutants could explain the complexity of over-accumulated lipids in LE/Ls despite the presence of binding motifs for cholesterol but not other lipids in NPC1 and NPC2. In other words, LE/L tubulation could be the more global function for NPC genes that are impaired in NPC disease.

Loss of Purkinje cells is a signature feature of NPC disease. Our newly generated StARD9 knock-out mice display a similar progressive loss of Purkinje cells that correlates with the onset 25 and severity of neurodegeneration symptoms. Also, like mouse models of NPC disease, StARD9 KO mice develop tremors, ataxia, loss of grip strength and abnormal walking gait in that order. Interestingly, a human patient with mutations in StARD9 was reported recently. Many of the symptoms in this patient resemble late-stage NPC disease, including neurodegeneration defects and epilepsy. This further supports the potential overlap in function between StARD9 and NPC genes.

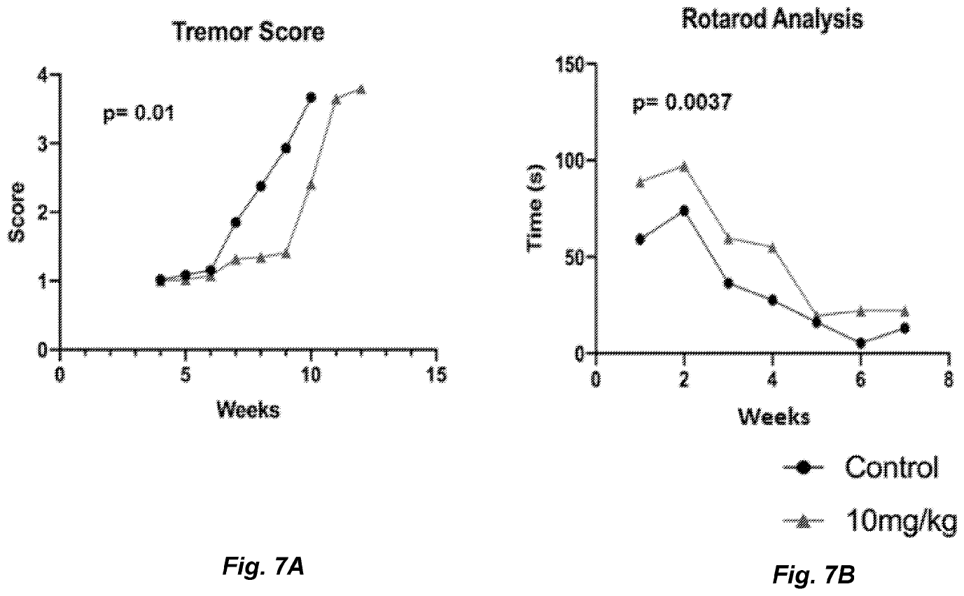

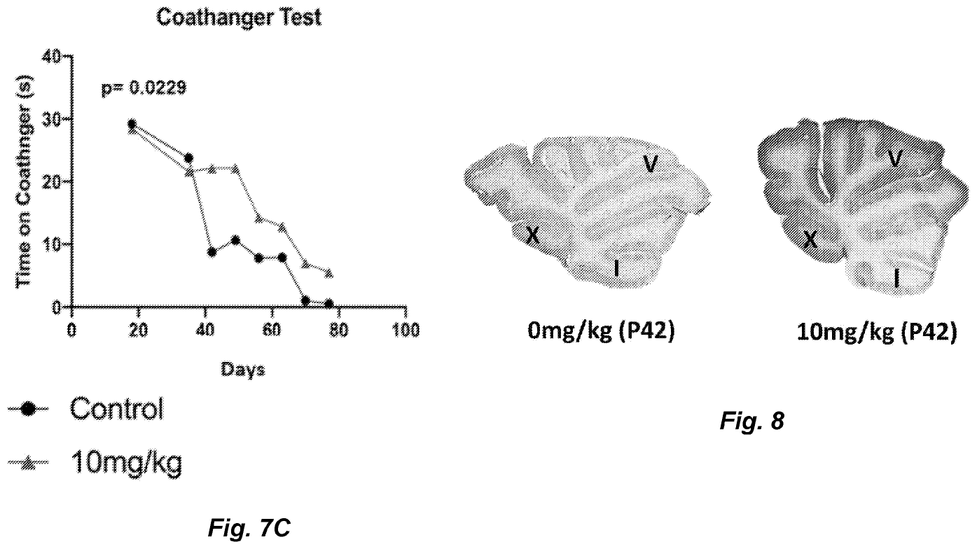

StARD9 is a novel LE/L kinesin that contributes to both bidirectional motility of LE/Ls and projection of plus end-directed membrane tubules from the surface of LE/Ls (See Sterling, FIG. 7). Because mutations in NPC1 impact both activities, we propose that NPC1 functions with StARD9 to regulate LE/L membrane dynamics. Loss of this regulation has the potential to explain the defects in LE/L cholesterol transport in NPC disease.

### Reagents:

Chemical and standard reagents including filipin and NBD-cholesterol were obtained from Sigma-Aldrich, St. Louis, MO. Lysotracker and fluorescent dextran were obtained from Life Technologies (Grand Island, NY). ShRNA constructs were obtained from SA Biosciences (Valencia, CA). Antibodies against tubulin, EGFP, mCherry have been described previously (Whyte et al., J. Cell Biol. 183, 819-834 (2008); Towns et al., Cell Motil. Cytoskeleton 66, 80-89 (2009)). Antibodies against StARD9 were obtained from Sigma-Aldrich. Antibodies against GM2 were provided by Konstantin Dobrenic (Albert Einstein University). Site-directed mutagenesis utilized the Phusion kit (New England Biolabs, Ipswich, MA). Antibodies to NPC1 (Cat. #ab134113), LAMP-1 (Cat. #abH4Δ3) and cathepsin B (Cat. #ab6313) were obtained from ABCAM (Boston, MA). Filipin (f9765) was obtained from Sigma Aldrich. NBD-cholesterol (N1148) was obtained from Invitrogen (Waltham, MA). BacMAM-ER (C10590) was obtained from Thermo-Fisher (Waltham, MA). Nocodazole (M1404) and taxol (T7402) were obtained from Sigma-Aldrich. The sequences of oligonucleotides used in this study are provided in Table 3 (See Sterling, Table 3).

### Plasmid Constructs:

The original NPC1 construct was obtained from Dr. T. Y. Chang (Dartmouth University) and subcloned into pCIneo placing the EGFP sequence at the C-terminus using standard methods. A similar approach was used to tag the C-terminus of NPC1 with mCherry. The NPC1 (I1061T) mutant construct was generated by PCR-based mutagenesis. ShRNA constructs co-expressing GFP as a transfection reporter were obtained from SA Biosciences. Construct (276) anneals to the StARD9 sequence at base pair 8138 and was identified as the most potent in StARD9 depletion. An EST clone encoding the C-terminal ˜1800 A.A. of StARD9 (KIAA1300) was obtained from Kazusa DNA Research Institute, Chiba, Japan (Nagase et al., DNA Res. 7, 65-73 (2000)). The remaining portions of StARD9 were cloned by RT-PCR from human cerebellum RNA (Agilent Technologies Inc., Wilmington, DE) and human liver mRNA (Life Technologies).

### Cell Lines:

Fixed and live-cell imaging was performed on COS-7 cells grown in Dulbecco's Modified Eagle's Medium (DMEM) supplemented with 10% fetal bovine serum, 1% L-glutamine, and penicillin/streptomycin (Sigma-Aldrich). Transfections were performed using either Lipofectamine (Life Technologies), X-treme Gene DNA Transfection reagent (Roche, Indianapolis, IN) or Nanofect (Allstem, Richmond, CA). J774A.1 mouse macrophages were provided and cultured in the same media. Cells were tested for contamination using PCR-based test kits (Sartorius, Inc.; Cat #SMB95-1001).

### LC/MSMS Analysis:

Purified NPC1-EGFP-containing membranes were subjected to reduction and alkylation, digestion with trypsin or Endoproteinase GluC (New England Biolabs). Samples were desalted by ZipTip (Millipore, Billerica, MA) according to manufacturers' instructions and separated on a nanoAcquity UHPLC system (Waters Billerica, MA) prior to MS-MS analysis. MS/MS-MS analysis was performed on an Amazon Ion trap instrument (Bruker Daltronics, Billerica, MA) running in DDA mode. Acquisition and processing were performed in the Mass Spectrometry and Proteomics Facility (MSPF) at The University of Notre Dame. Spectra were converted to .mgf files (MS-MS peak lists) using Data Analysis (Bruker) and subjected to database search with Mascot against the human Uniprot database. (downloaded 10-15-2009) MS tolerances were set to +/−1.5 Da for MS1 and 1.7 Da for MS2. Due to the small search space, traditional FDR was not employed as it is not accurate for small-n datasets. A mascot score of 40 and two peptides uniquely matching were used for ID cutoff.

### LC-MRM Analysis:

MRM transitions were selected and monitored on a 5500 QTrap coupled with an Eksigent 2D NanoUHPLC (AB Sciex) running chromatography and gradient as described in references above. Three or four transitions (as indicated) were selected for each peptide and six peptides were selected for monitoring for STARD9, two peptides for LAMP1, and two proteotypic peptides were observed for NPC1. Peak integration and mapping were performed in Skyline.

### Isolation of NPC1-Containing Membranes for MS/MS Analysis:

A stable cell line in COS-7 cells was generated using the pCI-neo (NPC1-EGFP) construct described above by standard methods. This cell line was expanded for biochemical isolation of NPC1-containing membranes.

The cell pellet from 5-150 cm tissues culture plates was subjected to hypotonic/mechanical lysis and brought to 2M sucrose for separation in a 0.5M/1.3M/2M sucrose gradient. The resulting membrane fraction at the 0.5M/1.3M interface was further subjected to immunoprecipitation with anti-NPC1 antibodies to isolate the NPC1-containing membrane fraction. This fraction was subjected to tryptic digestion without membrane solubilization to focus on the cytoplasmic face of the membranes. A protein profile was obtained using MASCOT analysis of MS/MS results.

### Preparation of Purified Phagosomes:

J774A.1 mouse macrophages were fed fish-gelatin-coated 0.8 mm latex beads (Sigma) and allowed to internalize beads overnight. Treated cultures were subjected to mechanical lysis with a motorized pestle, brought to 2.0M sucrose and loaded under a 0.5M/1.3M sucrose step gradient. After 100 kG centrifugation in a swinging-bucket rotor, the phagosomes at the 0.5M/1.3M interface were isolated and for biochemical extraction. Extracted samples were processed for MS/MS analysis after solubilization by standard methods.

### PNGase F Analysis:

Protein samples were digested from mouse cerebellums or PANC-1 cells with RIPA buffer and mechanical homogenization. Samples were incubated with 5% SDS and 1M DTT for 5 min. at 95° C. then 5 min. at room temperature. Then the samples were incubated with 1×PBS, 10% NP-40 and PNGase F for three hours at 37° C. Control reactions omitted PNGase F. The samples were then subjected to immunoblotting. Multiple exposures of the StARD9 blot are presented in Sterling, Sup. FIG. 7. The blot for NPC1 is included for comparison but is not new to this study.

### Timelapse Imaging:

Microscopy experiments were performed on a Zeiss Axiovert 200M (Thornwood, NY) run by Metamorph software (Sunnyvale, CA). Fixed and live cell imaging data was collected using either a Photometrics Coolsnap HQ or Cascade 1k CCD camera (Tucson, AZ). Timelapse imaging of cells expressing NPC1-EGFP and mCherry-StARD9 utilized a Dual-view camera (Photometrics) adapter to allow simultaneous acquisition of green and red channels at each timepoint. Kymographs were generated in Metamorph using a 1p m slice from each frame.

### Generation of StARD9(−/−) Mice:

CRISPR/Cas9 reagents were obtained from Transposagen (Lexington, KY) and provided to the Transgenic Core Facility (Purdue University) for injection into 1-cell C57Bl/6 mouse embryos. Resulting mice were genotyped by PCR to detect deletions of the StARD9 gene. Resulting PCR products were cloned into plasmids and subjected to sequencing to define the precise junctions around the guide-RNA sequence-annealing sites. StARD9 (+/−) mice were expanded for breeding to produce StARD9 (+/+), (+/−) and (−/−) mice in accordance with IACUC compliance.

### Mouse Husbandry:

Mice were housed socially in temperature-controlled room with a 12 hour light-dark cycle. They were fed a chow diet. All caretaking and experiments were approved by IACUC. Mice were euthanized between 2-6 months, or if they weighed 30% less than their littermates. Tissue from these mice was used for histology studies.

### StARD9 Mouse Behavioral Analysis:

Mice were binned in to three categories labeled 1-3. 1 corresponds to least severe, 2 as moderate, and 3 as severe. A composite score for symptom severity was developed from two measurements: coat hanger test and tremors. For the coat hanger test, mice were placed at the center of a standard coat hanger (3 mm diameter) by their front paws and observed for 30 secs. Following observation, the mice were scored as follows: 1—remains on the coat hanger for at least 30 secs; 2—falls off the coat hanger between 10-30 secs; 3—falls off the coat hanger in less than 10 secs. The second observation was tremors. Mice were observed for two minutes by an individual who did not know the genotype and were scored based on the following: 1—no tremors; 2—minor tremors lasting for less than ten seconds; 3—major tremors lasting for more than ten seconds. The two scores were averaged and are displayed in Table 2.

### Cerebellum Calbindin Staining:

Cerebellums were dissected and placed in 4% paraformaldehyde for 24 hours at 4° C. After fixing, the tissue was placed in 20% sucrose at 4° C. for 12 hours. After being set in VWR Premium Frozen Section Compound (VWR, 95-57-838), 11 μm sections were made using a Leica CM1850 Cryostat and placed on VWR Superfrost Plus Slides (VWR, 48311-703). Sections were dried at room temperature for 30 minutes before staining. They were washed twice for ten minutes in 1×PBS at room temperature. Sections were incubated in blocking solution (PBS with 1% BSA, 1.5% donkey serum, and 0.02% saponin) for 2 and a half hours at room temperature. Then the sections were incubated overnight at 4° C. in primary solution (1:3000 Calbindin, Sigma C9848). On the second day, the sections were washed four times in wash solution (PBS with 0.01% saponin) for ten minutes. Then the sections were incubated for 1 hour at room temperature with biotinylated secondary antibodies (1:250 Biotinylated GAM, Vector BA-9200). The sections were then washed five times for ten minutes in wash solution. Then they were incubated in Vectastain ABC kit (Vector PK-4000) for 1 hour at room temperature. The sections were then washed four times for ten minutes in 1×PBS. Then the sections were incubated in Peroxidase Substrate Kit DAB (Vector SK-4100) for ten minutes. They were then rinsed twice with 1×PBS and mounted with Prolong Gold (Invitrogen, F9765) and stored at 4° C. until imaging. Imaging was performed using a Leica S APO microscope. Color intensity was measured using ImageJ.

### Cultured Cell Experiments:

The variable being measured, the number of independent experiments and the number of cells from which data were collected are specified in the legend for each figure. Data are presented as violin plots generated in Graphpad PRISM. Statistical significance was determined using either a Student's T-test or ANOVA analysis depending experimental design.

### StARD9 Mouse Experiments:

Three mice from per genotype were analyzed per experiment.

Images were analyzed using ImageJ for cell count and intensity. Means were calculated using Microsoft Excel and are presented as mean±SEM. Student's t-test was used to compare cell count and mean intensity. Sequence Accession Code for All StARD9 Sequences is: NM_020759

### Example 2. Treatment of NPC Disease

NPC1 is required for STING silencing by the lysosome and the loss of NPC1 prevents STING silencing in NPC mutant mice. This may explain the Purkinje cell loss and immune response observed in the cerebellum in NPC disease. This function for NPC1 is completely independent of a role in cholesterol transport and unknown by the NPC community until now. This finding has the potential to revolutionize our thinking about treatments for NPC disease.

To narrow down the regions of NPC1 responsible for STING interactions at the lysosome, we have generated truncation constructs in NPC1 (FIG. 1) that retain lysosomal targeting function identify sub-sequences in NPC1 that rescue NPC1 function and could be used for gene therapy constructs with adenovirus.

Applicant proposes that NPC1 plays an essential role in stimulating lysosomal tubulation as a mechanism to transport cholesterol from the lysosome to the ER. Using NPC1 mutants, we demonstrated that NPC1 is required for lysosomal tubulation and that loss of tubulation in NPC1 mutants prevents cholesterol transport to the ER. We also determined that a novel function for NPC1 and NPC2 is to carry out the “Sequential Transfer Mechanism” of cholesterol transport (FIG. 5). In this model, NPC2 selects cholesterol out of the complex mixture of steroids in LDL particles and provides it to the NTD of NPC1. NPC1 then transfers that cholesterol to the SSD of NPC1 which functions to deliver cholesterol to the cytoplasmic side of the membrane.

We propose that the target for this cholesterol is the StART domain of StARD9. We discovered StARD9 as a novel lysosomal kinesin responsible for driving lysosomal tubulation. Consistent with this model, StARD9-driven lysosomal tubulation is dependent on uptake of LDL and the presence of intact NPC1 (FIGS. 2, 3, 4, and 6). We propose that a major function for NPC2 and NPC1 is to transport LDL-derived cholesterol to StARD9 as a signaling molecule to activate tubulation. This is consistent with a new StARD9 knock-out mouse line we would generate a feedback loop that couples LDL-uptake to intracellular cholesterol transport.

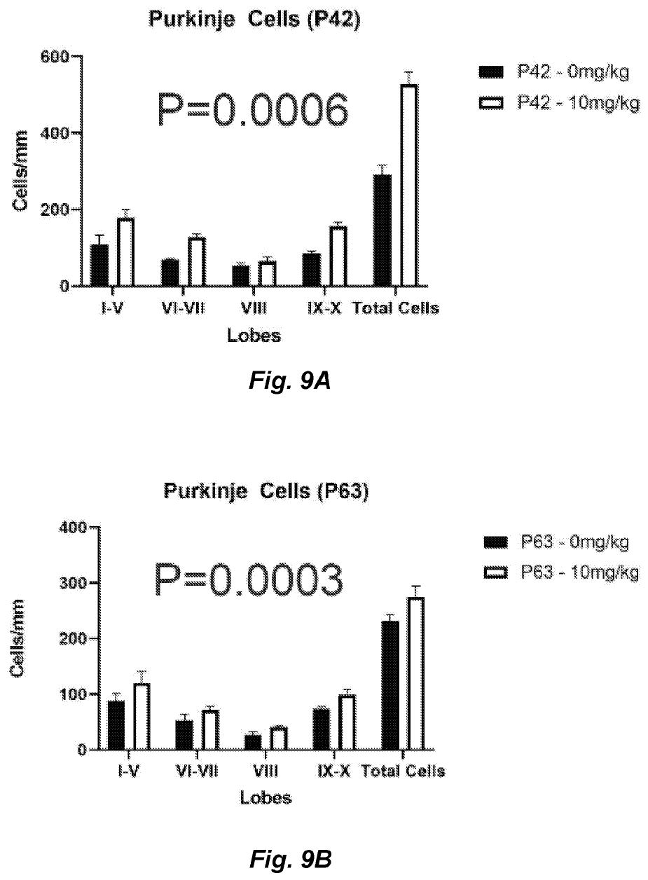

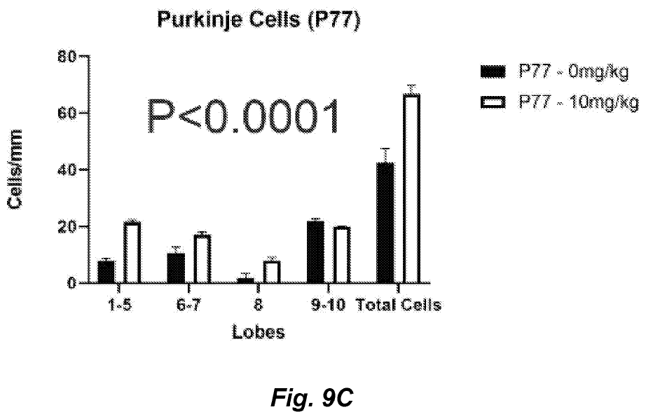

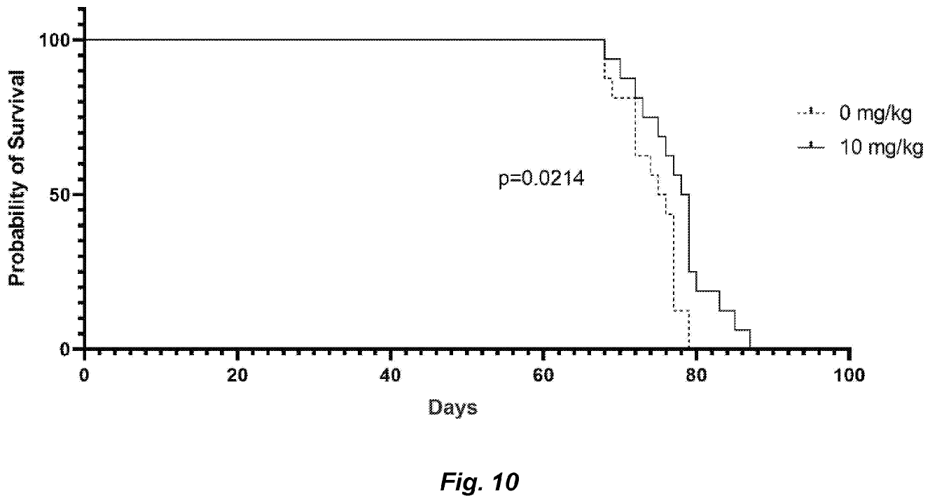

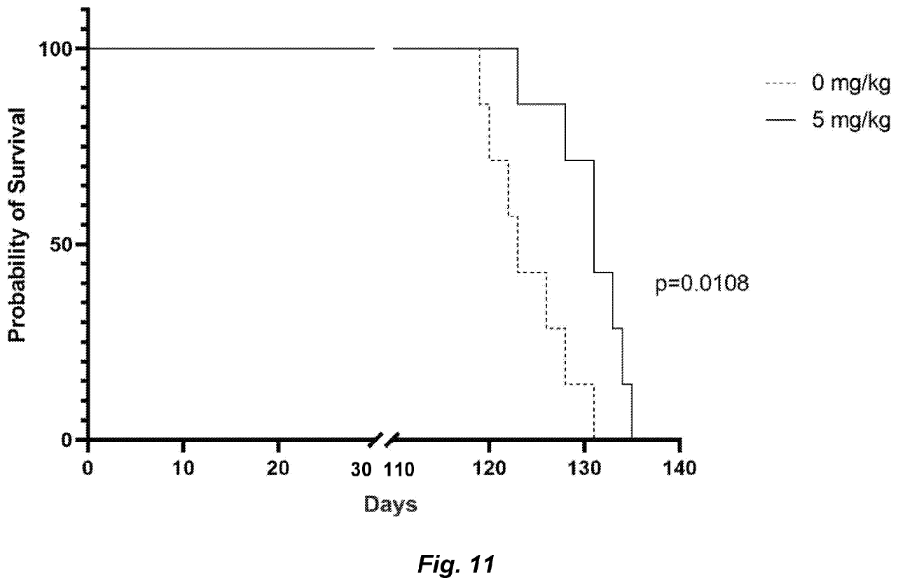

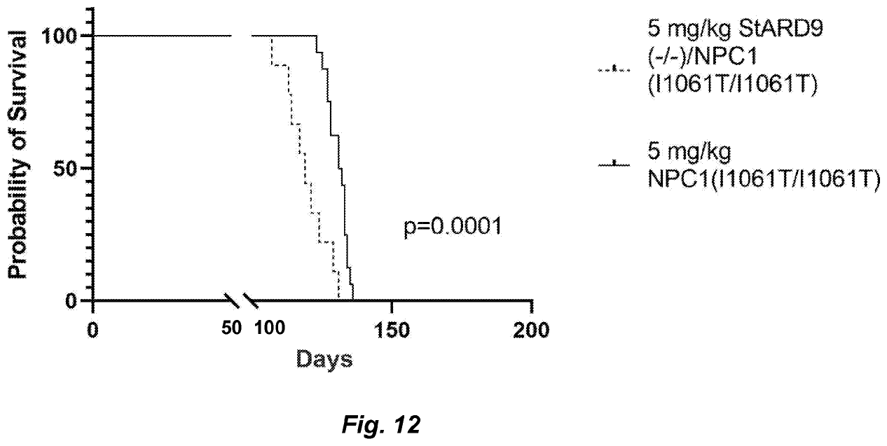

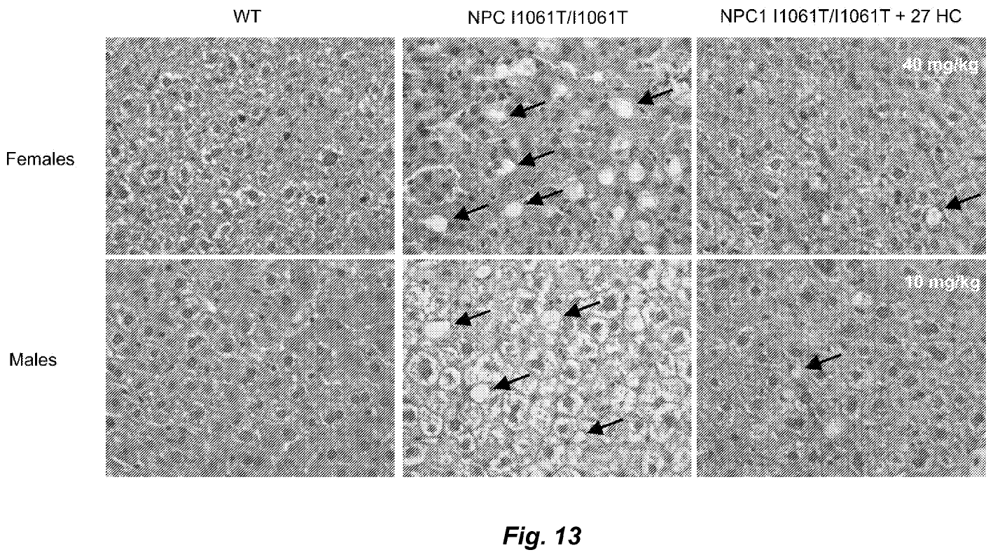

Interestingly, when we tested cholesterol and individual oxysterols as signaling molecules for StARD9, we found that 27-hydroxycholesterol (27-HC) was the most potent candidate. This suggests that a nuance to the “Sequential Transfer Mechanism” is that NPC1 and NPC2 select 27-HC out of the complex mixture in LDL and transport his molecule specifically. To test this idea, we treated either NPC1 mutant (null or I1061T) or StARD9 mutant (null or truncation mutant) cells with 27-HC in a cocktail that delivers it into the cytoplasm independent of receptor-mediated endocytosis. 27-HC rescued lysosomal tubulation in NPC1 mutant but not StARD9 mutant cells (FIG. 6). Next, we treated NPC1(−/−) mice with 27-HC by i.p. delivery. 27-HC treatment delayed the onset of symptoms, reduced the severity of symptoms, preserved Purkinje cells (FIGS. 7A-C) and extended the lifespan of the mice compared to controls with vehicle alone (FIGS. 8, 9A-C, and 10). Treating NPC1 (I1061T/I1061T) mice with 27-HC by oral gavage delayed the onset of symptoms, reduced the severity of symptoms and extended the lifespan of the mice compared to controls with vehicle alone (FIG. 11), however this treatment worked better in males than females. Analyzing the livers of these mice, we observed that the highest dose we use was not sufficient in females compared to males. Interestingly, when we treated mice that were both NPC1 and StARD9 mutant, the benefit of the 27-HC treatment was lost (FIGS. 12, 13). This suggests that StARD9 is the target for 27-HC treatment and that NPC1 is required for delivery of 27-HC to StARD9 under normal conditions.

We further propose that NPC1 is required for two functions in cells. The first is cholesterol transport from the lysosome to ER. If this activity is impaired, cells are “starved” of cholesterol in the ER. This has consequences for cholesterol metabolism and activates STING. Second, NPC1 in the lysosome is the receptor for STING that is required for STING silencing. In the absence of NPC1, STING remains active after initial activation (Priming) but also amplifies the responses including apoptosis. The dual roles of NPC1 are consistent with results across the NPC field. First, this explains why our StARD9 (−/−) mice are less severe than NPC1 (−/−) mice. NPC1(−/−) mice cause STING activation and impair STING silencing. StARD9 (−/−) mice only cause STING activation but do not impair STING silencing. Second, NPC1 (−/−) mice are more severe than NPC1 (I1061 T/I1061 T) mice because the NPC1 (10161 T) mice must reduce STING activation and retain some STING silencing compared to nulls. These treatments with 27-HC provide a statistically-significant improvement for NPC1 mutant mice.

Applicants further investigated which treatment Strategy with 27-hydroxycholesterol produces the most optimal disease outcomes in mouse models of NPC disease. These investigations include varying at least four aspects of a treatment scheme, including dosage amount, treatment frequency, delivery cocktail and in utero treatment.

Studies have tested treatment 1× per week and 3× per week. However, from pharmacokinetic studies, it is known that 27-HC levels are only retained for 1-2 days. We will increase to daily treatment by oral gavage and measure serum levels of 27-HC to confirm maintenance of therapeutic levels. We selected treatment doses (1 mg/kg, 5 mg/kg, 10 mg/kg) based on the solubility of 27-HC in our delivery cocktail (PBS, DMSO, PEG-400). We will increase to higher doses by adding higher levels of DMSO to allow treatment with 20 mg/kg and 40 mg/kg.

We anticipate an increased benefit from treatment on a daily basis because the pharmacokinetics inform us about how long 27-HC remains in the blood (-100 ng/ml for 12 hours). For reference, children treated with Miglustat are dosed 3× per day. Higher doses of 27-HC and alternative cocktails are expected to reveal which parameters are more important for success. Preliminary data reveals that mice harboring a transgene for Cyp27a, the gene responsible for 27-HC production, exhibit blood levels of 27-HC that are 5× higher than normal (˜500 ng/ml). When crossed with NPC1(−/−) mice, they observe a significant decrease in NPC disease symptoms and an extension of lifespan (personal communication). This provides independent validation of the benefits associated with treatment with 27-HC. Finally, we expect improved outcomes for mice treated from conception over waiting until weaning (P21). This is because there is such a long window of brain development and maturation from conception to P21, that any developmental aspect of NPC disease would be well underway before we begin treatment at weaning.

### Example 3. Pharmaceutical Dosage Forms

The following formulations illustrate representative pharmaceutical dosage forms that may be used for the therapeutic or prophylactic administration of a compound of a formula described herein, a compound specifically disclosed herein (an oxysterol or 27-hydroxycholesterol), or a pharmaceutically acceptable salt or solvate thereof (hereinafter referred to as ‘Compound X’):

| (i) Tablet 1 | mg/tablet |
|---|---|
| ‘Compound X’ | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

| (ii) Tablet 2 | mg/tablet |
|---|---|
| ‘Compound X’ | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| ‘Compound X’ | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/mL) | mg/mL |
|---|---|
| ‘Compound X’ (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |

| (v) Injection 2 (10 mg/mL) | mg/mL |
|---|---|
| ‘Compound X’ (free acid form) | 10.0 |
| Monobasic sodium phosphate | 0.3 |
| Dibasic sodium phosphate | 1.1 |
| Polyethylene glycol 400 | 200.0 |
| 0.1N Sodium hydroxide solution | q.s. |
| (pH adjustment to 7.0-7.5) | |
| Water for injection | q.s. ad 1 mL |

| (vi) Aerosol | mg/can |
|---|---|
| ‘Compound X’ | 20 |
| Oleic acid | 10 |
| Trichloromonofluoromethane | 5,000 |
| Dichlorodifluoromethane | 10,000 |
| Dichlorotetrafluoroethane | 5,000 |

| (vii) Topical Gel 1 | wt. % |
|---|---|
| ‘Compound X’ | 5% |
| Carbomer 934 | 1.25% |
| Triethanolamine (pH adjustment to 5-7) | q.s. |
| Methyl paraben | 0.2% |
| Purified water | q.s. to 100 g |

| (viii) Topical Gel 2 | wt. % |
|---|---|
| ‘Compound X’ | 5% |
| Methylcellulose | 2% |
| Methyl paraben | 0.2% |
| Propyl paraben | 0.02% |
| Purified water | q.s. to 100 g |

| (ix) Topical Ointment | wt. % |
|---|---|
| ‘Compound X’ | 5% |
| Propylene glycol | 1% |
| Anhydrous ointment base | 40% |
| Polysorbate 80 | 2% |
| Methyl paraben | 0.2% |
| Purified water | q.s. to 100 g |

| (x) Topical Cream 1 | wt. % |
|---|---|
| ‘Compound X’ | 5% |
| White bees wax | 10% |
| Liquid paraffin | 30% |
| Benzyl alcohol | 5% |
| Purified water | q.s. to 100 g |

| (xi) Topical Cream 2 | wt. % |
|---|---|
| ‘Compound X’ | 5% |
| Stearic acid | 10% |
| Glyceryl monostearate | 3% |
| Polyoxyethylene stearyl ether | 3% |
| Sorbitol | 5% |
| Isopropyl palmitate | 2% |
| Methyl Paraben | 0.2% |
| Purified water | q.s. to 100 g |

These formulations may be prepared by conventional procedures well known in the pharmaceutical art. It will be appreciated that the above pharmaceutical compositions may be varied according to well-known pharmaceutical techniques to accommodate differing amounts and types of active ingredient ‘Compound X’. Aerosol formulation (vi) may be used in conjunction with a standard, metered dose aerosol dispenser. Additionally, the specific ingredients and proportions are for illustrative purposes. Ingredients may be exchanged for suitable equivalents and proportions may be varied, according to the desired properties of the dosage form of interest.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

What is claimed is:

1. A method of treating Nieman-Pick Type C (NPC) disease in a subject in need thereof comprising administering to a subject an effective amount of:
an oxysterol; and
a polypeptide that binds to a Stimulator of Interferon Gene (STING) protein;
wherein the oxysterol stimulates lysosomal tubulation and the polypeptide sequesters the STING protein to a lysosomal membrane for degradation, thereby treating NPC disease.

2. The method of claim 1, wherein the oxysterol comprises one or more of 20α-hydroxycholesterol, 22(R)-hydroxycholesterol, 22(S)-hydroxycholesterol; 24(R)-hydroxycholesterol, 24(S)-hydroxycholesterol, 25-hydroxycholesterol, and 27-hydroxycholesterol.

3. The method of claim 1, wherein the oxysterol is 27-hydroxycholesterol (27-HC).

4. The method of claim 1, wherein the polypeptide comprises Nieman-Pick C1 protein (NPC1).

5. The method of claim 1, wherein the polypeptide comprises a STING binding domain of NPC1.

6. The method of claim 5, wherein the STING binding domain of the NPC1 comprises:
amino acids 1-24 and 90-1278 of SEQ ID NO: 1;
amino acids 1-90 and 153-1278 of SEQ ID NO: 1;
amino acids 1-155 and 216-1278 of SEQ ID NO: 1;
amino acids 1-297 and 344-1278 of SEQ ID NO: 1,
amino acids 1-1170 and 1262-1278 of SEQ ID NO: 1;
amino acids 1-24, 614-821, and 1267-1278 of SEQ ID NO: 1; or
amino acids 1-24 and 1099-1278 of SEQ ID NO: 1.

7. The method of claim 1, further comprising administering to the subject an effective amount of a recombinant polynucleotide, wherein the recombinant polynucleotide comprises a nucleotide sequence encoding the polypeptide that binds to a STING protein.

8. The method of claim 7, further comprising administering to the subject an effective amount of a recombinant virus, wherein the recombinant virus is selected from the group consisting of a lentivirus, an adenovirus, an adeno-associated virus, and a retrovirus; wherein the recombinant virus comprises the recombinant polynucleotide encoding the polypeptide that binds to a STING protein.

9. The method of claim 1, wherein the oxysterol is orally administered, and optionally, wherein the oxysterol comprises a mixture of a buffering solution, a penetration enhancer, a polysorbate, and a polyethylene glycol.

10. The method of claim 9 wherein the buffering solution is phosphate buffered saline, the polysorbate is polysorbate-80, and the polyethylene glycol is polyethylene glycol-400.

11. The method of claim 9, wherein the pentation enhancer is one or more selected from the group consisting of dimethyl sulfoxide (DMSO), dimethyl formamide (DMF), dihydrolevoglucosenone, γ-butyrolactone (GBL), N-methyl-2-pyrrolidone (NMP), dimethylacetamide (DMAc), and ethanol.

12. The method of claim 1, wherein the oxysterol is administered to the subject in a dose of about 0.1 mg/kg to about 25 mg/kg, or about 1 mg/kg to about 15 mg/kg.

13. The method of claim 1, wherein the polypeptide is administered by injection or intravenous infusion; and the oxysterol and the polypeptide are administered sequentially or concurrently.

14. A method of treating a subject having Nieman-Pick Type C (NPC) disease comprising:
administering to the subject an effective amount of:
a first composition comprising 27-hydroxycholesterol, phosphate buffered saline, polysorbate-80, polyethylene glycol-400, and a penetration enhancer;
a second composition comprising a recombinant virus, wherein the recombinant virus comprises a polynucleotide having a nucleotide sequence encoding a polypeptide of Nieman-Pick C1 protein (NPC1) that binds to a Stimulator of Interferon Gene (STING) protein;
wherein the first composition is administered orally, and the second composition is administered intravenously, intramuscularly, or subcutaneously,
wherein the 27-hydroxycholesterol stimulates lysosomal tubulation and the polypeptide of NPC1 sequesters the STING protein to a lysosomal membrane for degradation, thereby treating the NPC disease.

15. The method of claim 14, wherein the polypeptide comprises a STING binding domain of NPC1 comprises: amino acids 1-24, and 90-1278 of SEQ ID NO: 1; amino acids 1-90 and 153-1278 of SEQ ID NO: 1; amino acids 1-155 and 216-1278 of SEQ ID NO: 1; amino acids 1-297 and 344-1278 of SEQ ID NO: 1; amino acids 1-1170 and 1262-1278 of SEQ ID NO: 1; amino acids 1-24, 614-821, and 1267-1278 of SEQ ID NO: 1; or amino acids 1-24 and 1099-1278 of SEQ ID NO: 1.

16. The method of claim 14, wherein the recombinant virus is a lentivirus, an adenovirus, an adeno-associated virus, or a retrovirus.

17. A method of treating Nieman-Pick Type C (NPC) disease in a subject comprising administering to the subject an effective amount of 27-hydroxycholesterol and a pharmaceutical acceptable carrier, wherein the 27-hydroxycholesterol stimulates lysosomal tubulation, thereby treating NPC disease, wherein the pharmaceutically acceptable carrier comprises phosphate buffered saline, a penetration enhancer, polysorbate-80, and polyethylene glycol-400.

18. The method of claim 17, wherein the 27-hydroxycholesterol is administered to the subject in a dose of about 1 mg/kg to about 15 mg/kg, or optionally, in a dose of about 8 mg/kg to about 12 mg/kg.

19. The method of claim 18, wherein the 27-hydroxycholesterol is orally administered.


SEQUENCE LISTING (4 sequences)

SEQ ID NO: 1 - protein, 1278 aa, Homo sapiens
MTARGLALGLLLLLLCPAQVFSQSCVWYGECGIAYGDKRYNCEYSGPPKPLPKDGYDLVQELCPGFFFGNVSLCCDVRQLQTLKDNLQLPLQFLSRCPSCFYNLLNLFCELTCSPRQSQFLNVTATEDYVDPVTNQTKTNVKELQYYVGQSFANAMYNACRDVEAPSSNDKALGLLCGKDADACNATNWIEYMFNKDNGQAPFTITPVFSDFPVHGMEPMNNATKGCDESVDEVTAPCSCQDCSIVCGPKPQPPPPPAPWTILGLDAMYVIMWITYMAFLLVFFGAFFAVWCYRKRYFVSEYTPIDSNIAFSVNASDKGEASCCDPVSAAFEGCLRRLFTRWGSFCVRNPGCVIFFSLVFITACSSGLVFVRVTTNPVDLWSAPSSQARLEKEYFDQHFGPFFRTEQLIIRAPLTDKHIYQPYPSGADVPFGPPLDIQILHQVLDLQIAIENITASYDNETVTLQDICLAPLSPYNTNCTILSVLNYFQNSHSVLDHKKGDDFFVYADYHTHFLYCVRAPASLNDTSLLHDPCLGTFGGPVFPWLVLGGYDDQNYNNATALVITFPVNNYYNDTEKLQRAQAWEKEFINFVKNYKNPNLTISFTAERSIEDELNRESDSDVFTVVISYAIMFLYISLALGHMKSCRRLLVDSKVSLGIAGILIVLSSVACSLGVFSYIGLPLTLIVIEVIPFLVLAVGVDNIFILVQAYQRDERLQGETLDQQLGRVLGEVAPSMFLSSFSETVAFFLGALSVMPAVHTFSLFAGLAVFIDFLLQITCFVSLLGLDIKRQEKNRLDIFCCVRGAEDGTSVQASESCLFRFFKNSYSPLLLKDWMRPIVIAIFVGVLSFSIAVLNKVDIGLDQSLSMPDDSYMVDYFKSISQYLHAGPPVYFVLEEGHDYTSSKGQNMVCGGMGCNNDSLVQQIFNAAQLDNYTRIGFAPSSWIDDYFDWVKPQSSCCRVDNITDQFCNASVVDPACVRCRPLTPEGKQRPQGGDFMRFLPMFLSDNPNPKCGKGGHAAYSSAVNILLGHGTRVGATYFMTYHTVLQTSADFIDALKKARLIASNVTETMGINGSAYRVFPYSVFYVFYEQYLTIIDDTIFNLGVSLGAIFLVTMVLLGCELWSAVIMCATIAMVLVNMFGVMWLWGISLNAVSLVNLVMSCGISVEFCSHITRAFTVSMKGSRVERAEEALAHMGSSVFSGITLTKFGGIVVLAFAKSQIFQIFYFRMYLAMVLLGATHGLIFLPVLLSYIGPSVNKAKSCATEERYKGTERERLLNF

SEQ ID NO: 2 - DNA, 4759 bp, Homo sapiens
  source 1..4759: mol_type = other DNA
cttcctgaccggcgcgcgcagcctgctgccgcggtcagcgcctgctcctgctcctccgctcctcctgcgcggggtgctgaaacagcccggggaagtagagccgcctccggggagcccaaccagccgaacgccgccggcgtcagcagccttgcgcggccacagcatgaccgctcgcggcctggcccttggcctcctcctgctgctactgtgtccagcgcaggtgttttcacagtcctgtgtttggtatggagagtgtggaattgcatatggggacaagaggtaaattgcgaatattctggcccaccaaaaccattgccaaaggatggatatgacttagtgcaggaactctgtccaggattcttctttggcaatgtcagtctctgttgtgatgttcggcagcttcagacactaaaagacaacctgcagctgcctctacagtttctgtccagatgtccatcctgtttttataacctactgaacctgttttgtgagctgacatgtagccctcgacagagtcagtttttgaatgttacagctactgaagattatgttgatcctgttacaaaccagacgaaaacaaatgtgaaagagttacaatactacgtcggacagagttttgccaatgcaatgtacaatgcctgccgggatgtggaggccccctcaagtaatgacaaggccctgggactcctgtgtgggaaggacgctgacgcctgtaatgccaccaactggattgaatacatgttcaataaggacaatggacaggcaccttttaccatcactcctgtgttttcagattttccagtccatgggatggagcccatgaacaatgccaccaaaggctgtgacgagtctgtggatgaggtcacagcaccatgtagctgccaagactgctctattgtctgtggccccaagccccagcccccacctcctcctgctccctggacgatccttggcttggacgccatgtatgtcatcatgtggatcacctacatggcgtttttgcttgtgttttttggagcattttttgcagtgtggtgctacagaaaacggtattttgtctccgagtacactcccatcgatagcaatatagctttttctgttaatgcaagtgacaaaggagaggcgtcctgctgtgaccctgtcagcgcagcatttgagggctgcttgaggcggctgttcacacgctgggggtctttctgcgtccgaaaccctggctgtgtcattttcttctcgctggtcttcattactgcgtgttcgtcaggcctggtgtttgtccgggtcacaaccaatccagttgacctctggtcagcccccagcagccaggctcgcctggaaaaagagtactttgaccagcactttgggcctttcttccggacggagcagctcatcatccgggcccctctcactgacaaacacatttaccagccatacccttcgggagctgatgtaccctttggacctccgcttgacatacagatactgcaccaggttcttgacttacaaatagccatcgaaaacattactgcctcttatgacaatgagactgtgacacttcaagacatctgcttggcccctctttcaccgtataacacgaactgcaccattttgagtgtgttaaattacttccagaacagccattccgtgctggaccacaagaaaggggacgacttctttgtgtatgccgattaccacacgcactttctgtactgcgtacgggctcctgcctctctgaatgatacaagtttgctccatgacccttgtctgggtacgtttggtggaccagtgttcccgtggcttgtgttgggaggctatgatgatcaaaactacaataacgccactgcccttgtgattaccttccctgtcaataattactataatgatacagagaagctccagagggcccaggcctgggaaaaagagtttattaattttgtgaaaaactacaagaatcccaatctgaccatttccttcactgctgaacgaagtattgaagatgaactaaatcgtgaaagtgacagtgatgtcttcaccgttgtaattagctatgccatcatgtttctatatatttccctagccttggggcacatgaaaagctgtcgcaggcttctggtggattcgaaggtctcactaggcatcgcgggcatcttgatcgtgctgagctcggtggcttgctccttgggtgtcttcagctacattgggttgcccttgaccctcattgtgattgaagtcatcccgttcctggtgctggctgttggagtggacaacatcttcattctggtgcaggcctaccagagagatgaacgtcttcaaggggaaaccctggatcagcagctgggcagggtcctaggagaagtggctcccagtatgttcctgtcatccttttctgagactgtagcatttttcttaggagcattgtccgtgatgccagccgtgcacaccttctctctctttgcgggattggcagtcttcattgactttcttctgcagattacctgtttcgtgagtctcttggggttagacattaaacgtcaagagaaaaatcggctagacatcttttgctgtgtcagaggtgctgaagatggaacaagcgtccaggcctcagagagctgtttgtttcgcttcttcaaaaactcctattctccacttctgctaaaggactggatgagaccaattgtgatagcaatatttgtgggtgttctgtcattcagcatcgcagtcctgaacaaagtagatattggattggatcagtctctttcgatgccagatgactcctacatggtggattatttcaaatccatcagtcagtacctgcatgcgggtccgcctgtgtactttgtcctggaggaagggcacgactacacttcttccaaggggcagaacatggtgtgcggcggcatgggctgcaacaatgattccctggtgcagcagatatttaacgcggcgcagctggacaactatacccgaataggcttcgccccctcgtcctggatcgacgattatttcgactgggtgaagccacagtcgtcttgctgtcgagtggacaatatcactgaccagttctgcaatgcttcagtggttgaccctgcctgcgttcgctgcaggcctctgactccggaaggcaaacagaggcctcaggggggagacttcatgagattcctgcccatgttcctttcggataaccctaaccccaagtgtggcaaagggggacatgctgcctatagttctgcagttaacatcctccttggccatggcaccagggtcggagccacgtacttcatgacctaccacaccgtgctgcagacctctgctgactttattgacgctctgaagaaagcccgacttatagccagtaatgtcaccgaaaccatgggcattaacggcagtgcctaccgagtatttccttacagtgtgttttatgtcttctacgaacagtacctgaccatcattgacgacactatcttcaacctcggtgtgtccctgggcgcgatatttctggtgaccatggtcctcctgggctgtgagctctggtctgcagtcatcatgtgtgccaccatcgccatggtcttggtcaacatgtttggagttatgtggctctggggcatcagtctgaacgctgtatccttggtcaacctggtgatgagctgtggcatctccgtggagttctgcagccacataaccagagcgttcacggtgagcatgaaaggcagccgcgtggagcgcgcggaagaggcacttgcccacatgggcagctccgtgttcagtggaatcacacttacaaaatttggagggattgtggtgttggcttttgccaaatctcaaattttccagatattctacttcaggatgtatttggccatggtcttactgggagccactcacggattaatatttctccctgtcttactcagttacatagggccatcagtaaataaagccaaaagttgtgccactgaagagcgatacaaaggaacagagcgcgaacggcttctaaatttctagccctctcgcagggcatcctgactgaactgtgtctaagggtcggtcggtttaccactggacgggtgctgcatcggcaaggccaagttgaacaccggatggtgccaaccatcggttgtttggcagcagctttgaacgtagcgcctgtgaactcaggaatgcacagttgacttgggaagcagtattactagatctggaggcaaccacaggacactaaacttctcccagcctcttcaggaaagaaacctcattctttggcaagcaggaggtgacactagatggctgtgaatgtgatccgctcactgacactctgtaaaggccaatcaatgcactgtctgtctctccttttaggagtaagccatcccacaagttctataccatatttttagtgacagttgaggttgtagatacactttataacattttatagtttaaagagctttattaatgcaataaattaactttgtacacatttttatataaaaaaacagcaagtgatttcagaatgttgtaggcctcattagagcttggtctccaaaaatctgtttgaaaaaagcaacatgttcttcacagtgttcccctagaaaggaagagatttaattgccagttagatgtggcatgaaatgagggacaaagaaagcatctcgtaggtgtgtctactgggttttaacttatttttctttaataaaatacattgttttcctaagttttggggttaccctatctgctttgagagacaaatacaaaagctaaatggaagaga

SEQ ID NO: 3 - DNA, 673 bp, Homo sapiens
  source 1..673: mol_type = other DNA
cgtagaacttaaaacagaccctggtccccacctcggggtttcctcgcgcccacgggctgcgctgcgcagctgggccagcaggtggccgaggctcccgcactttttttcccgggcctggccgcaccgccaccgcccaccccgcgctccgccccgccccgccccgccgcactcgcggatcgtcacacggtcaggccaccgcgcgtcaccgccccgggctctgctagaaagtgccggggtccagattcctttcaggtgactgggttgggaggagaagtcgctcacggtaattgtcggagccgaaataaacagagactccgcggccgggcgggaccttacccggaagtcccccagaacacgactacccttcgctgaaacctcgcccgccccctccaacccagagacgaaaagggaaagcatcacggccagaaaccgttggcacaactccacagatactctcccgggccgagccagactccataagtcccgcgcctggcccccggggattgcaggggctgaggagaagggcaacacggggaccttgaagcggggtcgcggcggcgccccagcccgggccagggagtcccggcagcggcacctcccagaaagggcggagccgacgacgccttcttccttcctgaccggcgcgcgcagtcctgctgccgcggtcagcgcc

SEQ ID NO: 4 - DNA, 363 bp, Mus musculus
  source 1..363: mol_type = other DNA
acttgtggactaagtttgttcgcatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgcgaggctgtgagcagccacagtgccctgctcagaagccccaagctcgtcagtcaagccggttctccgtttgcactcaggagcacgggcaggcgagtggcccctagttctgggggcag